(19) United States
(12) Patent Application Publication
(10) Pub. No.: US 2024/0087699 A1
(43) Pub. Date: Mar. 14, 2024

(54) GRAPHICAL USER INTERFACE FOR SURGICAL PERFORMANCE ASSESSMENT

(71) Applicant: INTUITIVE SURGICAL OPERATIONS, INC., Sunnyvale, CA (US)

(72) Inventors: Kristen Brown, Roswell, GA (US); Anthony Jarc, Johns Creek, GA (US); Yihan Bao, Norcross, GA (US); Xi Liu, Alpharetta, GA (US); Huan Phan, Belmont, CA (US); Linlin Zhou, Alpharetta, GA (US)

(21) Appl. No.: 18/556,581

(22) PCT Filed: Apr. 24, 2022
(86) PCT No.: PCT/US2022/026080

§ 371 Date: Oct. 20, 2023

Related U.S. Application Data
(60) Provisional application No. 63/180,452, filed on Apr. 27, 2021.

(51) Int. Cl. G16H 15/00; G16H 10/60; G16H 50/70

(52) U.S. Cl. CPC G16H 15/00; G16H 10/60; G16H 50/70

(57) ABSTRACT

Various of the disclosed embodiments provide Graphical User Interfaces (GUIs) for reviewing prior surgical procedures. Specifically, the GUI may allow a user, such as surgeon, to review sensor data, including video data, acquired during various of the surgeon's past surgical procedures. Some sensor data may be organized into metrics referred to herein as objective performance indicators (OPIs). Similarly, procedures may be discretized into specific tasks. By organizing and presenting data in OPI form at the task level, the GUI may facilitate efficient and coordinated review of the surgeon's progress over time across multiple procedures. In some embodiments, corresponding data from expert surgeons may also presented in the interface so that the user may gauge the surgeon's relative performance.

### CROSS-REFERENCE TO RELATED APPLICATIONS

This application claims the benefit of, and priority to, U.S. Provisional Application No. 63/180,452, filed upon Apr. 27, 2021, entitled “GRAPHICAL USER INTERFACE FOR SURGICAL PERFORMANCE ASSESSMENT” and which is incorporated by reference herein in its entirety for all purposes.

### TECHNICAL FIELD

Various of the disclosed embodiments relate to computer systems and computer-implemented methods for assessing surgical performances.

### BACKGROUND

During their initial surgical training, novice surgeons may benefit from the real-time presence of an experienced colleague by their side in the surgical theater. Such a mentor may comment upon the novice surgeon's choices in real-time, direct the novice surgeon to more efficient practices and protocols, as well as provide feedback regarding the novice surgeon's progress across surgeries. Unfortunately, such real-time monitoring and guidance cannot be provided indefinitely as the mentor's obligations to other novice surgeons, as well as the mentor's own surgical obligations, limit the mentor's ability to continue to provide such guidance. Thus, novice surgeons may have limited opportunities for ongoing education or feedback regarding their performance after their official training concludes. Even experienced surgeons may likewise have limited vehicles for improving their skills or appreciating how their performance compares to their peers. Surgeons operating independently rarely have opportunities to compare techniques or to discover differences in their practice methods. Hospital administrators and other analysts may similarly have a difficult time comparing surgeons' skill levels and assessing their decisions during surgery.

Fortunately, the introduction of improved sensors in the surgical theater, as well as the introduction of robotic surgical systems, have enabled granular, ongoing monitoring and assessment of a surgeon's performance over time. These tools may record video of the surgeon's operations (e.g., visual laparoscopic video, sonograms, infrared range-finder depth data, etc.), as well as various instrument values, such as laparoscopic tool positions, orientations, energy applications, operation duration, etc. during a surgery. In theory, this data could be used to guide the surgeon's development in a manner at least as effective, and possibly more effective, than that of the real-time mentor, as the surgeon could review this data at any time, and as many times, as the surgeon desired. Experienced surgeons may likewise compare sensor values from their performances with those of peers to infer, e.g., relative trends in their practices. Hospital administrators, insurers, technicians, and other analysts may likewise benefit from reviewing such collected data.

However, presenting such data in a meaningful and impactful manner is a difficult task. Few surgeons or administrators are able or willing to interpret raw sensor data values. Even if they were able to do so, the relation between those values and the surgeon's performance and progress over time would not often be readily manifest, let alone easily relatable to data acquired from other surgeons. Indeed, sensors may change over time, both in their operation and in their location in the theater. Ideally, it would be possible to recognize surgical progress despite the disparate character of different surgeries, sensors, surgical tasks, instruments, and the idiosyncratic approaches of individual surgeons. Additionally, the user would ideally be able to rapidly review and consider aspects of multiple past surgical procedures without being mired in an intractable morass of data. Accordingly, there exists a need for intuitive systems and interfaces which consolidate surgical data into a manner conducive to assessing or improving a variety of surgical techniques.

### BRIEF DESCRIPTION OF THE DRAWINGS

Various of the embodiments introduced herein may be better understood by referring to the following Detailed Description in conjunction with the accompanying drawings, in which like reference numerals indicate identical or functionally similar elements:

FIG. 1A is a schematic view of various elements appearing in a surgical theater during a surgical operation as may occur in relation to some embodiments;

FIG. 1B is a schematic view of various elements appearing in a surgical theater during a surgical operation employing a surgical robot as may occur in relation to some embodiments;

FIG. 2A is a schematic illustration of surgical data as may be acquired from a surgical theater in some embodiments;

FIG. 2B is a table of example tasks as may be used in conjunction with various disclosed embodiments;

FIG. 3 is a table of additional example tasks as may be used in conjunction with various disclosed embodiments;

FIG. 4A is a schematic diagram illustrating relations between various metrics and data structures as may be used in some embodiments;

FIG. 4B is a schematic depiction of an example raw data input, specifically, a forceps translational movement in three-dimensional space, as may be used to generate one or more objective performance indicator (OPI) metrics in some embodiments;

FIG. 4C is a schematic depiction of an example raw data input, specifically, a plurality of rotations in three-dimensional space about a plurality of forceps component axes, as may be used to generate one or more OPIs in some embodiments;

FIG. 4D is a pair of tables illustrating example OPI to skill and skill to task mappings as may be applied in some embodiments;

FIG. 5 is a schematic data flow diagram illustrating surgical data information capture and processing, as may occur in some embodiments;

FIG. 6 is a schematic window topology diagram illustrating navigation relations between various windows of a graphical user interface (GUI) application, as may be implemented in some embodiments;

FIG. 7 is a schematic computer screen layout depicting a Capture Case window as may be implemented in some embodiments;

FIG. 8 is a schematic computer screen layout depicting a Home window as may be implemented in some embodiments;

FIG. 9 is a flow diagram illustrating various operations in an example process for selecting recommended surgical datasets based upon one or more user datasets, as may be implemented in some embodiments;

FIG. 10 is a schematic computer screen layout depicting a My Videos window, as may be implemented in some embodiments;

FIG. 11 is a schematic computer screen layout depicting the My Videos window of FIG. 10 when a clinical task filter drop-down has been selected, as may be implemented in some embodiments;

FIG. 12 is a schematic computer screen layout depicting the My Videos window of FIG. 10 when a metric filter drop-down has been selected, as may be implemented in some embodiments;

FIG. 13 is a schematic computer screen layout depicting a My Metrics window with a scatter plot metric map, as may be implemented in some embodiments;

FIG. 14 is a schematic computer screen layout depicting the My Metrics window of FIG. 13 with a tabular metric map, as may be implemented in some embodiments;

FIG. 15 is a schematic computer screen layout depicting a video portion of a Procedure View window, as may be implemented in some embodiments;

FIG. 16 is a schematic computer screen layout depicting a video portion of a Procedure View window with an expert mirrored video, as may be implemented in some embodiments;

FIG. 17 is a schematic computer screen layout depicting a portion of Procedure View window with a scatter plot metric map, as may be implemented in some embodiments;

FIG. 18A is an extended schematic computer screen layout illustrating example relative positions of the video portion of the Procedure View window of FIG. 15 and the portion of the Procedure View window depicted in FIG. 17, as may be implemented in some embodiments;

FIG. 18B is an extended schematic computer screen layout illustrating example relative positions of the portion of the Procedure View window of FIG. 16 and the portion of the Procedure View window depicted in FIG. 17, as may be implemented in some embodiments;

FIG. 18C is an extended schematic computer screen layout illustrating example relative positions of a portion of a Procedure View window combining features from FIGS. 15 and 16 with the portion of the Procedure View window depicted in FIG. 17, as may be implemented in some embodiments;

FIG. 19 is a flow diagram illustrating various operations in an example “procedure-view drill-down” process for user procedures, as may be implemented in some embodiments;

FIG. 20 is a schematic computer screen layout depicting a portion of Procedure View window with a scatter plot metric map and intermediate selection panels, as may be implemented in some embodiments;

FIG. 21 is a flow diagram illustrating various operations in an example “procedure-view drill-down” process for both user and recommended procedures, as may be implemented in some embodiments;

FIG. 22 is a flow diagram illustrating various operations in an example “procedure-view drill-down” configuration process as may be implemented in some embodiments;

FIG. 23 is a table listing an example collection of OPIs, a description of each, and their relation to various skills and tasks;

FIG. 24 is a table listing an example collection of OPIs, a description of each, and their relation to various skills and tasks;

FIG. 25 is a table listing an example collection of OPIs, a description of each, and their relation to various skills and tasks;

FIG. 26 is a table listing an example collection of OPIs, a description of each, and their relation to various skills and tasks; and

FIG. 27 is a block diagram of an example computer system as may be used in conjunction with some of the embodiments.

The specific examples depicted in the drawings have been selected to facilitate understanding. Consequently, the disclosed embodiments should not be restricted to the specific details in the drawings or the corresponding disclosure. For example, the drawings may not be drawn to scale, the dimensions of some elements in the figures may have been adjusted to facilitate understanding, and the operations of the embodiments associated with the flow diagrams may encompass additional, alternative, or fewer operations than those depicted here. Thus, some components and/or operations may be separated into different blocks or combined into a single block in a manner other than as depicted. The embodiments are intended to cover all modifications, equivalents, and alternatives falling within the scope of the disclosed examples, rather than limit the embodiments to the particular examples described or depicted.

### DETAILED DESCRIPTION

### Example Surgical Theaters Overview

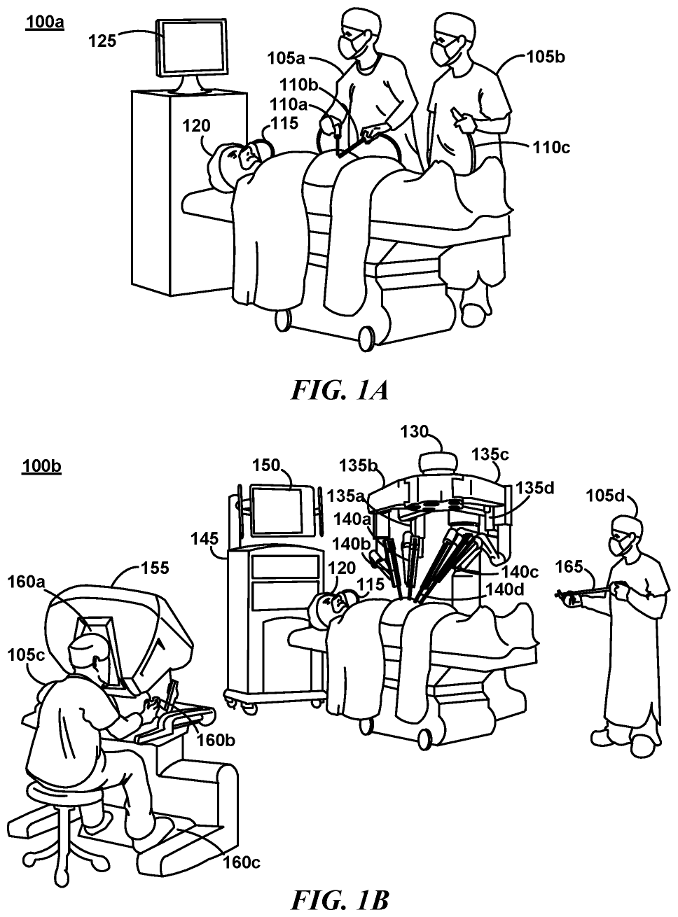

FIG. 1A is a schematic view of various elements appearing in a surgical theater 100a during a surgical operation as may occur in relation to some embodiments. Particularly, FIG. 1A depicts a non-robotic surgical theater 100a, wherein a patient-side surgeon 105a performs an operation upon a patient 120 with the assistance of one or more assisting members 105b, who may themselves be surgeons, physician's assistants, nurses, technicians, etc. The surgeon 105a may perform the operation using a variety of tools, e.g., a visualization tool 110b such as a laparoscopic ultrasound or endoscope, and a mechanical end effector 110a such as scissors, retractors, a dissector, etc.

The visualization tool 110b provides the surgeon 105a with an interior view of the patient 120, e.g., by displaying visualization output from a camera mechanically and electrically coupled with the visualization tool 110b. The surgeon may view the visualization output, e.g., through an eyepiece coupled with visualization tool 110b or upon a display 125 configured to receive the visualization output. For example, where the visualization tool 110b is an endoscope, the visualization output may be a color or grayscale image. Display 125 may allow assisting member 105b to monitor surgeon 105a's progress during the surgery. The visualization output from visualization tool 110b may be recorded and stored for future review, e.g., using hardware or software on the visualization tool 110b itself, capturing the visualization output in parallel as it is provided to display 125, or capturing the output from display 125 once it appears onscreen, etc. While two-dimensional video capture with visualization tool 110b may be discussed extensively herein, as when visualization tool 110b is an endoscope, one will appreciate that, in some embodiments, visualization tool 110b may capture depth data instead of, or in addition to, two-dimensional image data (e.g., with a laser rangefinder, stereoscopy, etc.). Accordingly, one will appreciate that it may be possible to apply the two-dimensional operations discussed herein, mutatis mutandis, to such three-dimensional depth data when such data is available.

A single surgery may include the performance of several groups of actions, each group of actions forming a discrete unit referred to herein as a task. For example, locating a tumor may constitute a first task, excising the tumor a second task, and closing the surgery site a third task. Each task may include multiple actions, e.g., a tumor excision task may require several cutting actions and several cauterization actions. While some surgeries require that tasks assume a specific order (e.g., excision occurs before closure), the order and presence of some tasks in some surgeries may be allowed to vary (e.g., the elimination of a precautionary task or a reordering of excision tasks where the order has no effect). Transitioning between tasks may require the surgeon 105a to remove tools from the patient, replace tools with different tools, or introduce new tools. Some tasks may require that the visualization tool 110b be removed and repositioned relative to its position in a previous task. While some assisting members 105b may assist with surgery-related tasks, such as administering anesthesia 115 to the patient 120, assisting members 105b may also assist with these task transitions, e.g., anticipating the need for a new tool 110c.

Advances in technology have enabled procedures such as that depicted in FIG. 1A to also be performed with robotic systems, as well as the performance of procedures unable to be performed in non-robotic surgical theater 100a. Specifically, FIG. 1B is a schematic view of various elements appearing in a surgical theater 100b during a surgical operation employing a surgical robot, such as a da Vinci™ surgical system, as may occur in relation to some embodiments. Here, patient side cart 130 having tools 140a, 140b, 140c, and 140d attached to each of a plurality of arms 135a, 135b, 135c, and 135d, respectively, may take the position of patient-side surgeon 105a. As before, the tools 140a, 140b, 140c, and 140d may include a visualization tool 140d, such as an endoscope, laparoscopic ultrasound, etc. An operator 105c, who may be a surgeon, may view the output of visualization tool 140d through a display 160a upon a surgeon console 155. By manipulating a hand-held input mechanism 160b and pedals 160c, the operator 105c may remotely communicate with tools 140a-d on patient side cart 130 so as to perform the surgical procedure on patient 120. Indeed, the operator 105c may or may not be in the same physical location as patient side cart 130 and patient 120 since the communication between surgeon console 155 and patient side cart 130 may occur across a telecommunication network in some embodiments. An electronics/control console 145 may also include a display 150 depicting patient vitals and/or the output of visualization tool 140d.

Similar to the task transitions of non-robotic surgical theater 100a, the surgical operation of theater 100b may require that tools 140a-d, including the visualization tool 140d, be removed or replaced for various tasks as well as new tools, e.g., new tool 165, introduced. As before, one or more assisting members 105d may now anticipate such changes, working with operator 105c to make any necessary adjustments as the surgery progresses.

Also similar to the non-robotic surgical theater 100a, the output from the visualization tool 140d may here be recorded, e.g., at patient side cart 130, surgeon console 155, from display 150, etc. While some tools 110a, 110b, 110c in non-robotic surgical theater 100a may record additional data, such as temperature, motion, conductivity, energy levels, etc. the presence of surgeon console 155 and patient side cart 130 in theater 100b may facilitate the recordation of considerably more data than is only output from the visualization tool 140d. For example, operator 105c's manipulation of hand-held input mechanism 160b, activation of pedals 160c, eye movement within display 160a, etc. may all be recorded. Similarly, patient side cart 130 may record tool activations (e.g., the application of radiative energy, closing of scissors, etc.), movement of end effectors, etc. throughout the surgery. In some embodiments, the data may have been recorded using an in-theater recording device, such as an Intuitive Data Recorder™ (IDR), which may capture and store sensor data locally or at a networked location.

### Data Overview

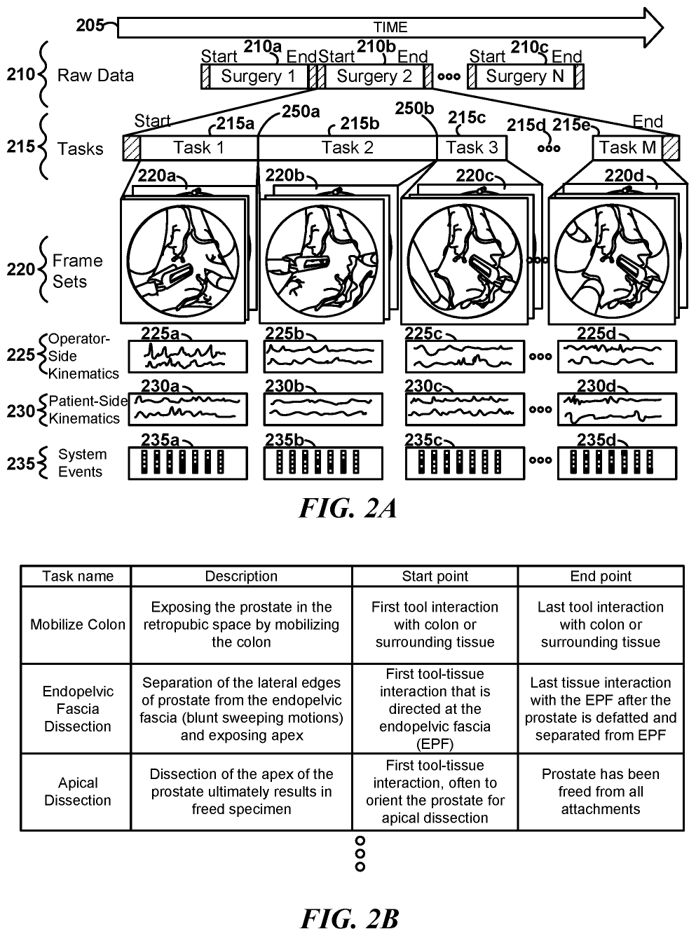

FIG. 2A is a schematic illustration of surgical data as may be acquired from a surgical theater in some embodiments. Specifically, a processing system may receive raw data 210, such as video from a visualization tool 110b or 140d comprising a succession of individual frames over time 205. In some embodiments, the raw data 210 may include video and system data from multiple surgical operations 210a, 210b, 210c, or only a single surgical operation.

As mentioned, each surgical operation may include groups of actions, each group forming a discrete unit referred to herein as a task. For example, surgical operation 210b may include tasks 215a, 215b, 215c, and 215e (ellipses 215d indicating that there may be more intervening tasks). Note that some tasks may be repeated in an operation or their order may change. For example, task 215a may involve locating a segment of fascia, task 215b involves dissecting a first portion of the fascia, task 215c involves dissecting a second portion of the fascia, and task 215e involves cleaning and cauterizing regions of the fascia prior to closure.

Each of the tasks 215 may be associated with a corresponding set of frames 220a, 220b, 220c, and 220d and device datasets including operator kinematics data 225a, 225b, 225c, 225d, patient-side device data 230a, 230b, 230c, 230d, and system events data 235a, 235b, 235c, 235d. For example, for video acquired from visualization tool 140d in theater 100b, operator-side kinematics data 225 may include translation and rotation values for one or more hand-held input mechanisms 160b at surgeon console 155. Similarly, patient-side kinematics data 230 may include data from patient side cart 130, from sensors located on one or more tools 140a-d, 110a, rotation and translation data from arms 135a, 135b, 135c, and 135d, etc. System events data 235 may include data for parameters taking on discrete values, such as activation of one or more of pedals 160c, activation of a tool, activation of a system alarm, energy applications, button presses, camera movement, etc. In some situations, task data may include one or more of frame sets 220, operator-side kinematics 225, patient-side kinematics 230, and system events 235, rather than all four.

One will appreciate that while, for clarity and to facilitate comprehension, kinematics data is shown herein as a waveform and system data as successive state vectors, one will appreciate that some kinematics data may assume discrete values over time (e.g., an encoder measuring a continuous component position may be sampled at fixed intervals) and, conversely, some system values may assume continuous values over time (e.g., values may be interpolated, as when a parametric function may be fitted to individually sampled values of a temperature sensor).

In addition, while surgeries 210a, 210b, 210c and tasks 215a, 215b, 215c are shown here as being immediately adjacent so as to facilitate understanding, one will appreciate that there may be gaps between surgeries and tasks in real-world surgical video. Accordingly, some video and data may be unaffiliated with a task or affiliated with a task not the subject of a current analysis. In some embodiments, these “non-task”/“irrelevant-task” regions of data may themselves be denoted as tasks during annotation, e.g., “gap” tasks, wherein no “genuine” task occurs.

The discrete set of frames associated with a task may be determined by the task's start point and end point. Each start point and each endpoint may, e.g., be itself determined by either a tool action or a tool-effected change of state in the body. Thus, data acquired between these two events may be associated with the task. For example, start and end point actions for task 215b may occur at timestamps associated with locations 250a and 250b respectively.

FIG. 2B is a table depicting example tasks with their corresponding start point and end points as may be used in conjunction with various disclosed embodiments. Specifically, data associated with the task “Mobilize Colon” is the data acquired between the time when a tool first interacts with the colon or surrounding tissue and the time when a tool last interacts with the colon or surrounding tissue. Thus any of frame sets 220, operator-side kinematics 225, patient-side kinematics 230, and system events 235 with timestamps between this start and end point are data associated with the task “Mobilize Colon”. Similarly, data associated the task “Endopelvic Fascia Dissection” is the data acquired between the time when a tool first interacts with the endopelvic fascia (EPF) and the timestamp of the last interaction with the EPF after the prostate is defatted and separated. Data associated with the task “Apical Dissection” corresponds to the data acquired between the time when a tool first interacts with tissue at the prostate and ends when the prostate has been freed from all attachments to the patient's body. One will appreciate that task start and end times may be chosen to allow temporal overlap between tasks, or may be chosen to avoid such temporal overlaps. For example, in some embodiments, tasks may be “paused” as when a surgeon engaged in a first task transitions to a second task before completing the first task, completes the second task, then returns to and completes the first task. Accordingly, while start and end points may define task boundaries, one will appreciate that data may be annotated to reflect timestamps affiliated with more than one task.

Additional examples of tasks include a “2-Hand Suture”, which involves completing 4 horizontal interrupted sutures using a two-handed technique (i.e., the start time is when the suturing needle first pierces tissue and the stop time is when the suturing needle exits tissue with only two-hand, e.g., no one-hand suturing actions, occurring in-between). A “Uterine Horn” task includes dissecting a broad ligament from the left and right uterine horns, as well as amputation of the uterine body (one will appreciate that some tasks have more than one condition or event determining their start or end time, as here, when the task starts when the dissection tool contacts either the uterine horns or uterine body and ends when both the uterine horns and body are disconnected from the patient). A “1-Hand Suture” task includes completing four vertical interrupted sutures using a one-handed technique (i.e., the start time is when the suturing needle first pierces tissue and the stop time is when the suturing needle exits tissue with only one-hand, e.g., no two-hand suturing actions occurring in-between). The task “Suspensory Ligaments” includes dissecting lateral leaflets of each suspensory ligament so as to expose ureter (i.e., the start time is when dissection of the first leaflet begins and the stop time is when dissection of the last leaflet completes). The task “Running Suture” includes executing a running suture with four bites (i.e., the start time is when the suturing needle first pierces tissue and the stop time is when the needle exits tissue after completing all four bites). As another example, the task “Rectal Artery/Vein” includes dissecting and ligating a superior rectal artery and vein (i.e. the start time is when dissection begins upon either the artery or the vein and the stop time is when the surgeon ceases contact with the ligature following ligation).

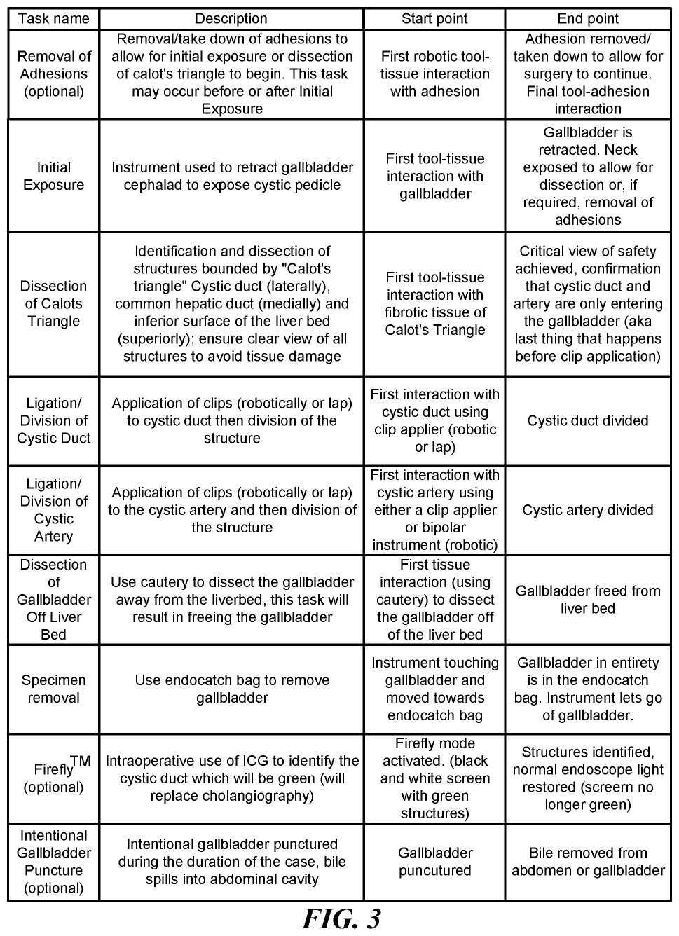

To provide yet additional example context, FIG. 3 is a table of additional example task definitions as may be used in conjunction with various disclosed embodiments. As indicated start and end points may determine what data is associated with a given task. One will appreciate that manual, human workflows, as well as computer-assisted or computer exclusive workflows may be used to segment surgical data into respective tasks based upon the start and end points. For example, some surgeons or their assistants may manually indicate task transitions during surgery, a robotic system may identify tasks during surgery from sensor data, a human annotator may identify tasks by manually inspecting the data, a post-processing system may automatically recognize patters in the data associated with distinct tasks, etc.

### Objective Performance Indicators—Application Overview

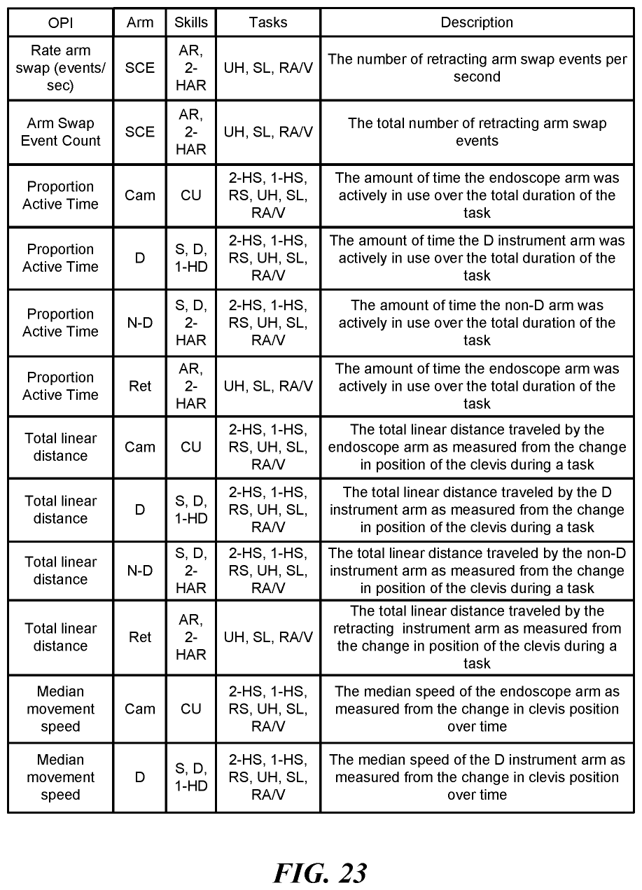

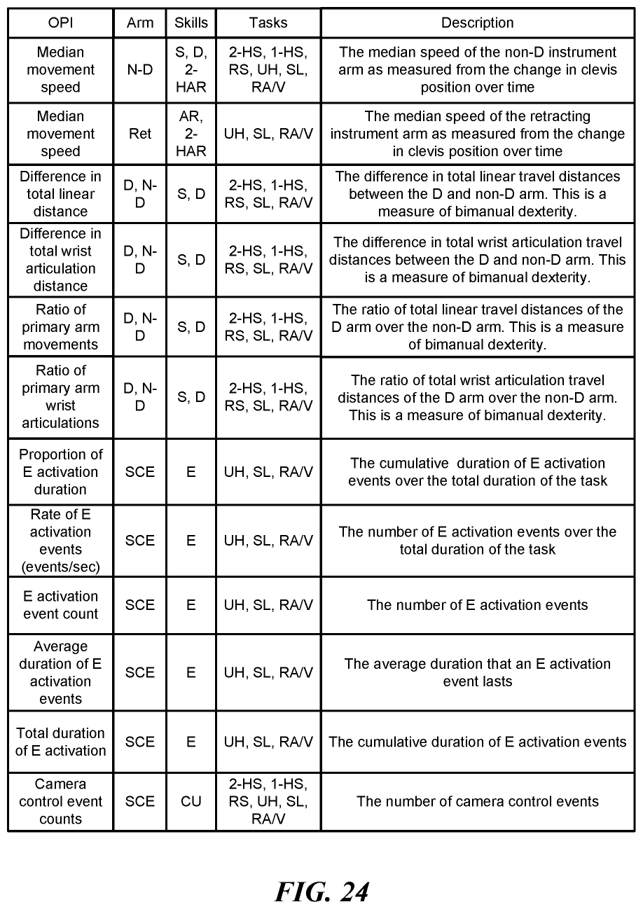

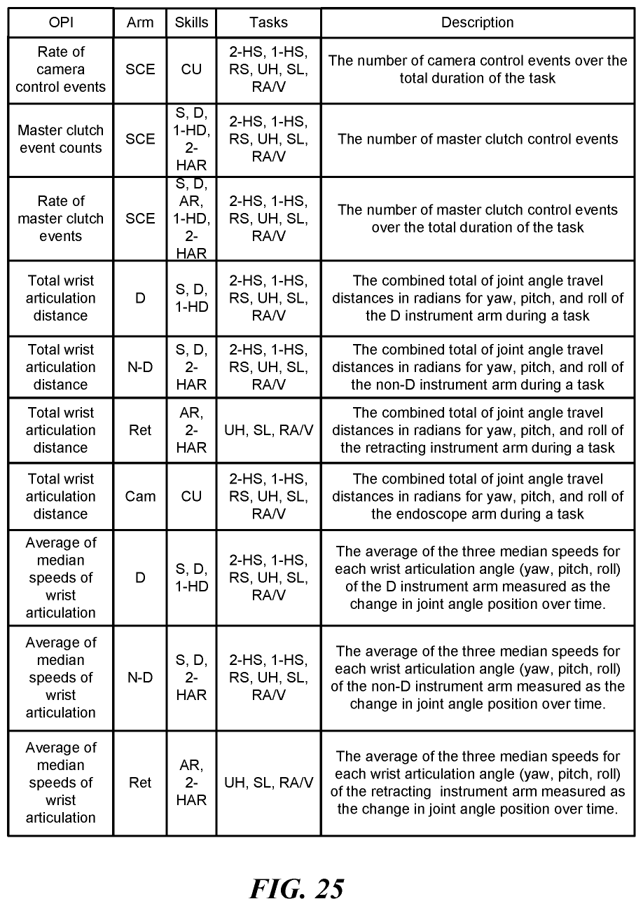

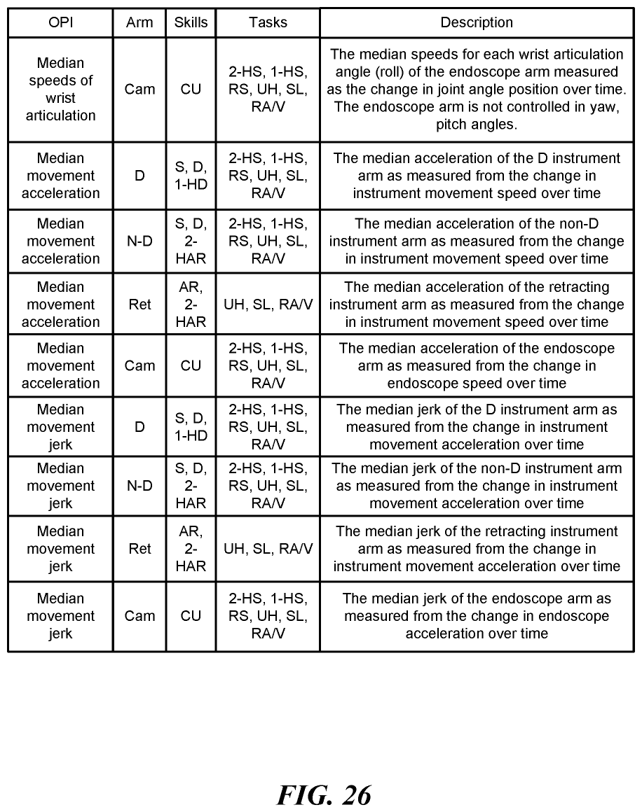

A surgeon's technical skills are an important factor in delivering optimal patient care. Unfortunately, many existing methods for ascertaining an operator's skill remain subjective, qualitative, or resource intensive. Various embodiments disclosed herein contemplate more effective surgical skill assessments by analyzing operator skills using OPIs, quantitative metrics generated from surgical data, which may be suitable for examining the operator's individual skill performance, task-level performance, as well as performance for the surgical operation as a whole. One will appreciate that OPIs may also be generated from other OPIs (e.g., the ratio of two OPIs may be considered an OPI), rather than taken directly from the data values. Skills are an action or a group of actions performed during a surgery recognized as influencing the efficiency or outcome of the surgery. Example OPIs are shown in the tables of FIGS. 23-26. While skills may be “defined” or represented by an initial assignment of OPIs (e.g., as suggested by an expert), often, it may suffice to simply consider OPIs directly for each task, and to ignore explicit consideration of any intermediate “skill” grouping.

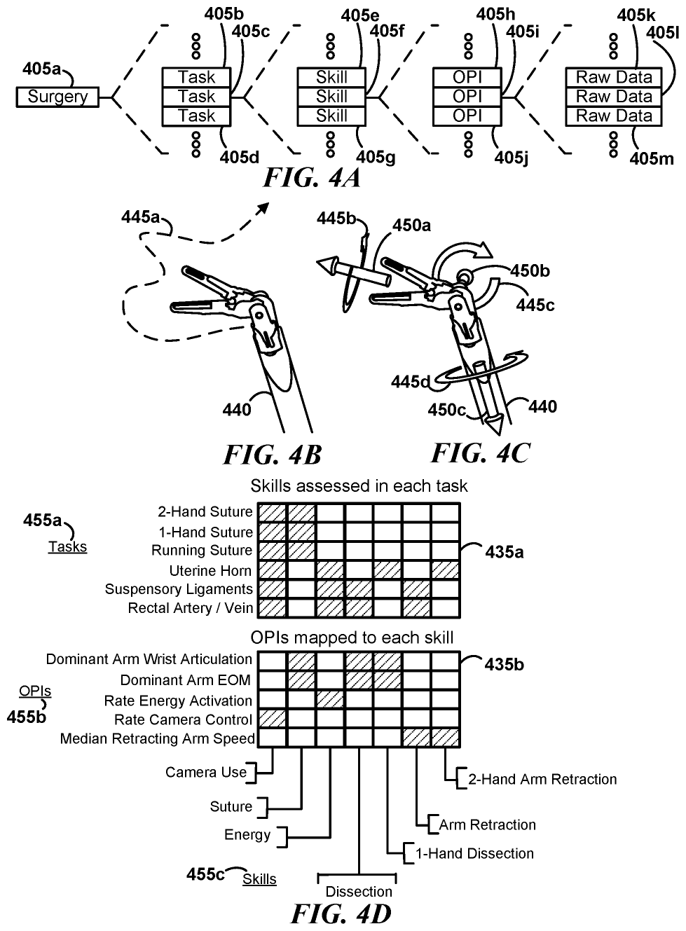

To facilitate understanding, FIG. 4A is a schematic diagram illustrating relations between various metrics and data structures as may be used in some embodiments. Specifically, a surgical operation 405a may consist of a plurality of tasks e.g., tasks 405b, 405c, and 405d. Each task may itself implicate a number skills. For example, task 405c may depend upon each of skills 405e, 405f, and 405g. In a similar manner, each skill may itself be assessed based upon one or more OPI metric values (though, again, OPI values may be directly related to tasks, without intervening skills, in some embodiments). For example, the skill 405f may be assessed by the OPI metrics 405h, 405i, and 405j. Each OPI metric may be derived from one or more raw data fields. For example, OPI metric 405i may depend upon raw data values 405k, 405l, and 405m (though, as mentioned, OPIs may also be derived from one another). Thus, care may be taken to divide the surgery into meaningful task divisions, to assess the skills involved in each task, to determine OPIs and relate them to the various skills (or simply to the tasks directly), and to define the OP Is from the available raw data.

As an example of raw data (specifically, kinematics data), FIG. 4B depicts a forceps 440's translational movement 445a in three-dimensional space, as may be used to generate one or more OPIs in some embodiments. FIG. 4C is an example raw data input, specifically, a plurality of rotations in three-dimensional space about a plurality of forceps component axes, as may be used to generate one or more OPIs in some embodiments. Forceps 440 may be able to rotate 445b, 445c, 445d various of its components about respective axes 450a, 450b, and 450c. The translations and rotations of FIGS. 4B and 4C may be captured in raw kinematics data over time, forming raw data values 405k, 405l, and 405m. OPI metric 405i may be a “forceps tip movement speed” OPI and may represent the speed of the forceps tip based upon the raw values 405k, 405l, and 405m (e.g., the OPI may infer the tip speed from a Jacobian matrix derived from the raw data of FIGS. 4B and 4C). OPI metric 405i may then be one of several OPI metrics used as part of a feature vector in a model to produce a skill score for skill 405f (or, again, a task score for task 405c). In some embodiments, collections of skill scores may then be used to assess the surgeon's performance of task 405c, and ultimately, by considering all the tasks, the surgeon's performance of the surgery 405a overall.

### Example OPI-Task Mapping

FIG. 4D is a pair of tables 435a, 435b illustrating example OPI to skill and skill to task mappings as may be contemplated in some embodiments (again, some embodiments may simply map OPIs to tasks directly, without explicitly acknowledging an intervening skill). With a plurality of skills 455c, shaded cells of table 435b indicate corresponding OPIs 455b. Similarly, table 435a indicates via shaded cells how tasks 455a may correspond to skills 455c.

For clarity, in the example correspondence shown in FIG. 4D, e.g., all six of the shown tasks depend upon the “Camera Use” skill, however, only the “Uterine Horn” task depends upon the “2-Hand Arm Retraction” skill. Similarly, the “Dominant Arm Wrist Articulation” OPI relates to the “Suture” skill. From these tables, one can also make transitive inferences, for example, that the “Rate Camera Control” OPI is relevant to the “Uterine Horn” task (as “camera use” is common to both in each of the tables). One will appreciate that more skills, tasks, and OPIs may apply than those shown in this example. Also note that a single skill may be applicable to multiple tasks. As mentioned, an initial OPI to skill correspondence, OPI to task correspondence, or skill to task correspondence may be determined by inspection or by consulting with an expert. Most example embodiments discussed herein will discuss OPIs directly in relation to tasks. However, the reader will appreciate that such task-OPI relations could be similarly expressed via an intermediate representation, such as, e.g., skills.

### Example Operator Assessment Processing Topology

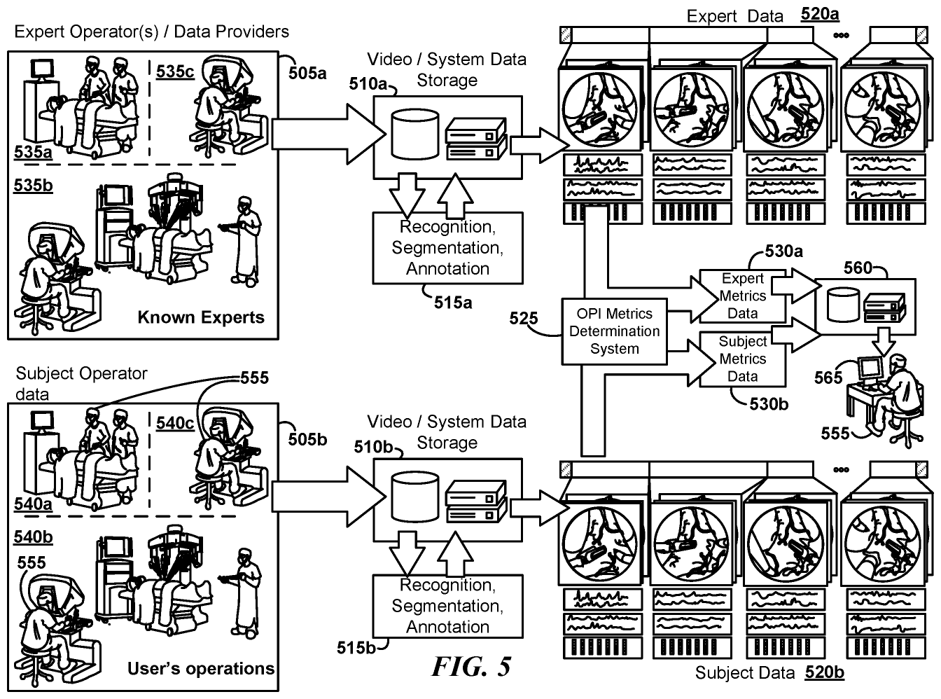

FIG. 5 is a schematic data flow diagram illustrating example surgical data information capture and processing, as may occur in some embodiments. Specifically, expert data 505a may be data acquired from one or more expert surgeons in real-world non-robotic surgery theaters 535a, real-world robotic surgery theaters 535b, and simulated operations 535c (though a robotic simulator is shown, one will appreciate that non-robotic surgeries may also be simulated, e.g. with appropriate dummy patient materials). One will appreciate that, in some embodiments, the data 505a is data from only one of real-world non-robotic surgery theaters 535a, real-world robotic surgery theaters 535b, or simulated operations 535c. “Experts” may be those surgeons with, e.g., more than 100 hours of experience performing a surgery, skill, or task, or those surgeons with case volumes exceeding a threshold, years of surgical experience generally, surgeons identified as being “experts” by hospitals or regulatory bodies, etc.

Similarly, data may be acquired for a “subject” surgeon 555, whose progress is to be evaluated alone, or relative to, the experts of dataset 505a. Accordingly, dataset 505b may be acquired from “subject” surgeon's 555 past surgeries, e.g., as data provided by real-world non-robotic surgery theaters 540a, real-world robotic surgery theaters 540b, and simulated operations 540c (again, though a robotic simulator is shown, one will appreciate that non-robotic surgeries may also be simulated, e.g. with appropriate dummy patient materials). Again, in some embodiments, dataset 505b may include only data from one, or some, of non-robotic surgery theaters 540a, real-world robotic surgery theaters 540b, and simulated operations 540c exclusively.

In some embodiments, expert dataset 505a and subject dataset 505b may be stored in data storages 510a and 510b, respectively, prior to consumption by OPI metrics determination system 525. In some embodiments data storages 510a and 510b may be the same data storage. In some embodiments, the data storages 510a and 510b may be offsite from the locations at which the data was acquired, e.g., in a cloud-based network server. Processing systems 515a and 515b may process the stored data in data storages 510a and 510b (e.g., recognizing distinct surgeries captured in the data stream, separating the surgeries recognized in the stream into distinct datasets, providing metadata annotations for the datasets, identifying and labeling tasks in the data, merely ensuring proper data storage without further action, etc.). In some embodiments, human annotators may assist, correct, or verify the results of processing systems 515a and 515b, e.g., adjusting task and surgery type classifications. In some embodiments processing systems 515a and 515b may be the same processing system.

Processed expert reference data 520a and subject data 520b in the data storages 510a and 510b may then be used by OPI metrics determination system 525 to determine performance metrics from the respective raw data. Specifically, system 525 may determine expert metrics data 530a from expert data 520a and system 525 may determine subject metrics data 530b from user data 520b, for each of the surgical operations reflected in the respective datasets. One will appreciate that system 525 may take a variety of forms, e.g., a hardware, software, or firmware system that may, e.g., simply map raw data values to OPI values in accordance with defined OPI functions, such as those appearing in the tables of FIGS. 23-26 herein.

One or more of the subject data 520b, subject metrics data 530b, expert data 520a, or expert metrics data 530a may then be presented via a computer system 560 to the subject surgeon 555, or another analyst, such as hospital administrator, at a local console 565 (such as a desktop computer, tablet computer, smartphone, etc.). For example, computer system 560 may be a server accessible via the Internet or a local network and local console 565 may be browser software running on a local computing device. In contrast, in some embodiments, computer system 560 and local console 565 may be the same system (e.g., as in a desktop application retrieving the OPI metrics and raw data from a local or network storage).

Thus, one will appreciate that the depicted topology is merely exemplary, and that various of the systems and operations may be physically or logically located otherwise than as shown here to facilitate the reader's comprehension. For example, in some embodiments, system 525 may be software logic within the system 560 determining OPI metric values from raw data upon console 565. Similarly, in some embodiments, system 525 may instead reside in the surgical theater, or in processing systems 515a and 515b.

While assessment of subject surgeon's 555 performance is described here relative to the performance of the experts in dataset 505a, one will appreciate that many of the disclosed embodiments may also apply to any surgeon among the set of experts associated with dataset 505a. That is, not only novice surgeons may benefit from the disclosed embodiments, but senior or expert surgeons wishing to improve their skills, or wishing to explore alternative surgical approaches, may likewise wish to compare their performance to their peers. Similarly, embodiments may thus not only educate surgeons, but may “educate educators” of surgeons, as when a teacher may now be able to more readily discern disparities between “ideal” performances and the performances of a cohort they intend to teach (e.g., it may make less sense to educate student surgeons on all tasks equally when it is clear from the data that there is only one task for which the students' performance radically departs from that of the experts).

### Example User Interface Window Topological Arrangement

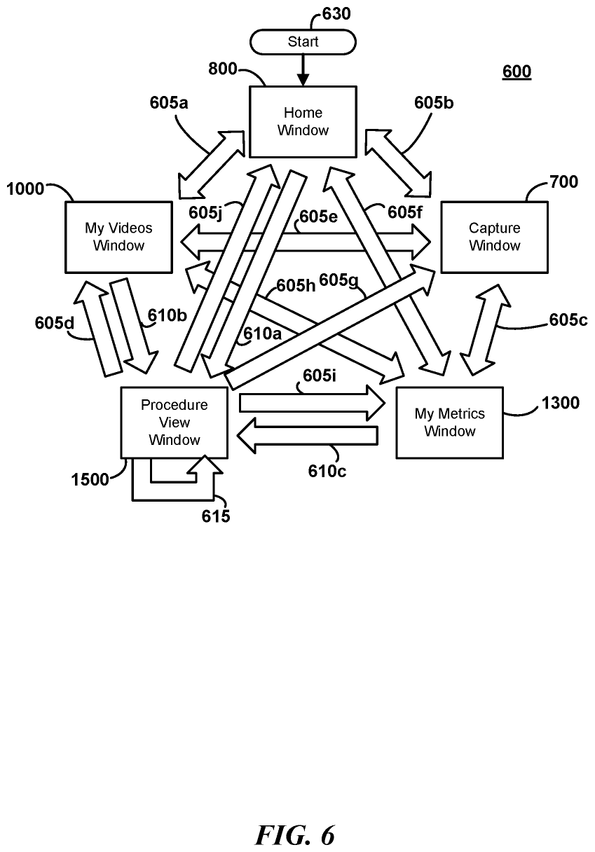

FIG. 6 is a window topology diagram illustrating navigation relations between various windows of a GUI application, as may be implemented in some embodiments. Specifically, a GUI presented on console 565 may allow surgeon 555, or other analyst, to navigate the raw and/or metrics data of the various surgical procedures. As a single, monolithic presentation of the data may overwhelm the analyst, the GUI may organize the information into a series of interfaces, such as browser windows. For clarity, a “window” may thus refer to a specific page, or portion of a page, of a website or a panel of a program, such as a desktop or tablet application. Where the windows are web pages, though pages may be acquired via page-specific URL requests, one will appreciate that this need not be the case in some embodiments, as Asynchronous JavaScript and XML (AJAX) and other development techniques may facilitate window transitions through partial modification of the display. While embodiments directed to browser windows are described herein to facilitate comprehension, one will appreciate that analogous presentations may be made, mutatis mutandis, in other interfaces, such as panes in a standalone desktop application, panels in a smartphone application, etc.

As indicated by the example topology 600, the GUI computer program on console 565 may facilitate transitions between a variety of windows. An analyst logging into the system may be first 630 presented with a Home window 800, which may provide summary statistics and updates to the analyst, as well as provide a central point for navigation. A Capture Case window 700 may allow the user to select appropriate recordation settings and hardware for a surgery they (or another user) are about to perform. My Metrics window 1300, as described in greater detail herein, provides an overview of the surgeon's performance across surgical procedures as represented in OPI values. My Videos window 1000 may provide a gallery from which the user may review and select specific surgical performances for review. The Procedure View window 1500, as described in greater detail herein, provides a granular depiction of the surgeon's performance during a specific surgery.

A navigation bar 715 present in each window may facilitate transitions 605a-j (e.g., load a new webpage, replace a window with new data, etc.) to and from each of the Home window 800, Capture Case window 700, My Videos window 1000, My Metrics window 1300, and from the Procedure View window 1500, as indicated. However, transitions to the Procedure View window 1500 may generally be effected by means other than the navigation bar 715 in the disclosed examples. Specifically, a transition 610a to the Procedure View window 1500 from the Home window 800 and the transition 610b from the My Videos window 1000 to the Procedure View window 1500 may be effected by the user selecting a pane depicting a particular surgical procedure. Transition 610c from the My Metrics window 1300 may be effected by making a selection in a metric map as descried herein. Distinguishing transitions to Procedure View window 1500 in this manner may facilitate the presentation of the surgical data in window 1500 based upon the origin and context of the source window. Procedure View window 1500 may also transition “to itself” 615 as the user iterates between procedures at a low, granular level, possibly examining a same task or metric across those procedures. Accordingly, as will be described in greater detail herein, various of these transitions may be constructed specifically to coordinate the presentation of information in the Procedure View window 1500 while minimizing disruption to the user's cognitive flow, whether the user is reviewing their own surgeries or, as in some embodiments, corresponding their review to that of related expert surgeon procedures.

### Example Capture Case Window

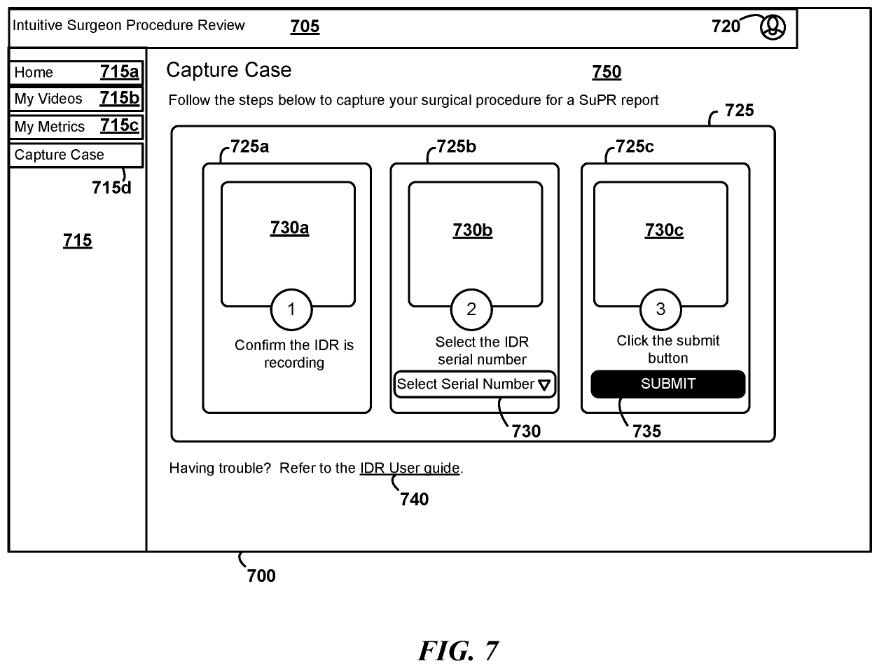

FIG. 7 is a schematic illustration of a computer screen depicting a Capture Case window 700 as may be implemented in some embodiments. Specifically, the window 700 may include a top bar region 705, a navigation bar region 715, and a region 750 depicting capture-specific features. Top bar region 705 may include a profile picture 720 associated with the user to, e.g., help the user verify that they are, in fact, reviewing data associated with their account (or with the surgeon they intend to review). Navigation bar region 715 may provide a series of selectors for navigating between windows, e.g., in accordance with the transitions of FIG. 6. Specifically, navigation bar region 715 may include an icon 715a presenting the Home window 800 when selected, an icon 715b presenting the My Videos window 1000 when selected, an icon 715c presenting the My Metrics window 1300 when selected, and an icon 715d presenting the Capture Case window 700 when selected.

The user may navigate to the Capture Case window 700 in anticipation of recording an upcoming surgical procedure. Accordingly, instructions may be provided within panel 725 for initiating the data recordation. For example, where the recordation device is an IDR, as discussed above, panel 725a may invite the user to confirm that the IDR is recording. Region 730a may depict a graphic inviting the user to inspect the IDR and verify recording. In some embodiments, however, the region 730a may provide an immediate indication of the data feed, such as video data, from the IDR. Often, there will be one IDR system for each surgical theater. Thus, in some large organizations, with multiple theaters or robotics systems, the surgeon may alternate between theaters, and consequently IDRs, across surgeries. Accordingly, a second panel 725b may invite the user to select the appropriate IDR via serial number from a drop down 730 of available IDR serial numbers. One will appreciate that the drop down 730 may be populated using a variety of mechanisms, e.g., real-time polling across a system network to detect IDR presence, consulting a record on a central server system, manual input from an Information Technology (IT) administrator, etc. Region 730b may depict an instructive graphic in some embodiments, such as the location of the serial number on the IDR. In some embodiments, however, region 730b may provide feedback for the available or selected IDR (e.g., location information, video feed data, etc.). Finally, panel 725c may invite the user to begin the recordation by clicking the submit button 735. Region 730c may provide an instructive graphic or may provide feedback regarding the recording state of the selected IDR. One will appreciate that not all the windows discussed herein need be presented on, or solely upon, the same system, e.g., console 565. For example, this window 700 may appear on console 565 as well as on one or more of displays 150, 160a, 125, etc. Window 700 may also invite the user to consult a guide via a HyperText Markup Language (HTML) uniform resource locator (URL) link 740 if they encounter issues.

In some embodiments, in addition to selecting the IDR serial number, the user may also input surgery metadata, such as the procedure type (e.g., “cholecystectomy”), surgeon ID (via the user's log-in/submit), patient data, etc. The system may likewise be integrated with a local staffing system or scheduling chart to collect this metadata. In some embodiments, in lieu of selecting an IDR via second panel 725b, subsequent logins by the user on an in-theater device (such as a console upon a robotic system) may facilitate metadata acquisition. For example, a network system may monitor logins on the robotic system (e.g., via electronics/control console 145), on console 155, and on a network system, associating a data capture with the appropriate account (e.g., when a same case ID appears on two or more systems). In some embodiments, window 700 is not included as part of the data review program on console 565, but appears only in the theater (e.g., on an interface to the IDR). The data may then be stored on, e.g., a network server or central storage for subsequent consideration by the GUI program at console 565.

### Example Home Window

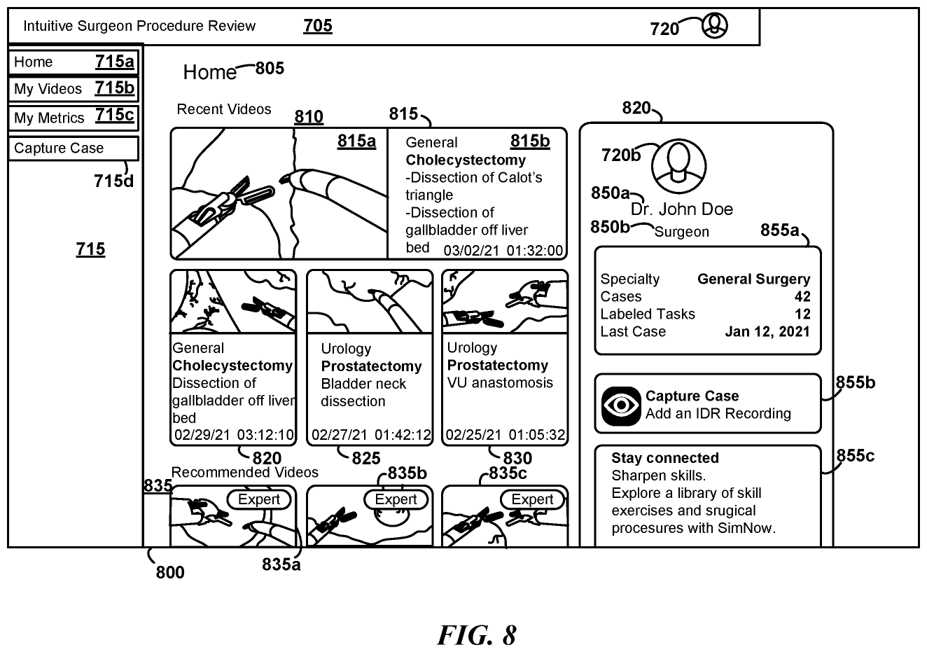

FIG. 8 is a schematic illustration of a computer screen depicting a Home window 800 as may be implemented in some embodiments. As was described with respect to FIG. 7, window 800 may also include navigation bar region 715 and top bar region 705. Label 805 may help confirm the window's identity to the user. A profile region 820 may provide information specific to the user. For example, an enlarged version 720b of the user's profile picture 720 may be presented, along with labels depicting the user's name 850a and designation 850b. Summary region 855a may indicate general user statistics, such as the user's specialty, number of recorded cases, tasks appearing in those cases, and the last case recorded for the user. A capture case button 855b may transition the user to Capture Case window 700, while a learning skills button 855c may invite the user to consider various learning materials.

Also shown in this example are recent videos region 810 and recommended videos 835 regions. The recent videos region 810 may present the subject surgeon's most recent procedure's information in a predominate pane 815, while previous surgery data captures may be presented in a chronologically decreasing order in panes 820, 825, and 830. As shown, each of the panes 815, 820, 825, and 830 may include both a video preview region (e.g., preview region 815a depicting, e.g., the output of an endoscope during the surgery) and a data summary region (e.g., summary region 815b) as well as indications of the date and duration of the surgery. Data summary regions (e.g., summary region 815b) may indicate the specialty (e.g., General, Urology, Cardiology, etc.) and procedure (e.g., Cholecystectomy, Prostatectomy, Cardiac Bypass, etc.), as well as a list of one or more tasks present in the surgery. In some embodiments, the video preview region is a static frame from the surgery. In some embodiments, hovering a mouse over the static frame may present a looping series of images to help the user appreciate the contents of the video recording. For example, the series of images may be successive frames in the video, or frames sampled at periodic (e.g., 10 minute) intervals from the video. In some embodiments, the preview region simply presents active video. In some embodiments, the panes may indicate whether the user has viewed/watched the videos previously to facilitate the user's comprehensive consideration.

The recommended videos region 835 may similarly include panes 835a, 835b, 835c with one or both of video preview regions and data summary regions. As indicated, the user may need to scroll down to view the entirety of panes 835a, 835b, 835c. When scrolling, the position of top bar region 705 and navigation bar region 715 may not change in the window (e.g., they may be designated as “fixed” position property in a Cascading Style Sheet (CSS) relative to the viewport, may reside in elements distinct from a scrolling element containing the recent videos region 810 and recommended videos region 835, etc.). In some embodiments, the recommended video panes may also indicate whether the user has viewed/watched the videos to facilitate comprehensive consideration.

### Example Recommended Dataset Selection Process

Recommended videos may be selected and ordered by the system based upon shared features with recent of the surgeon's performed cases (e.g., the cases depicted in recent videos region 810), with cases which the surgeon has demonstrated below-expert aptitude, cases with one or more better metrics scores as compared to a surgeon's selected case, etc. Accordingly, the recommended videos may be arranged based on chronology, the disparity of their metrics from the user's metrics for corresponding procedures, etc. The criteria by which datasets are recommended is referred to herein as “relevance.”

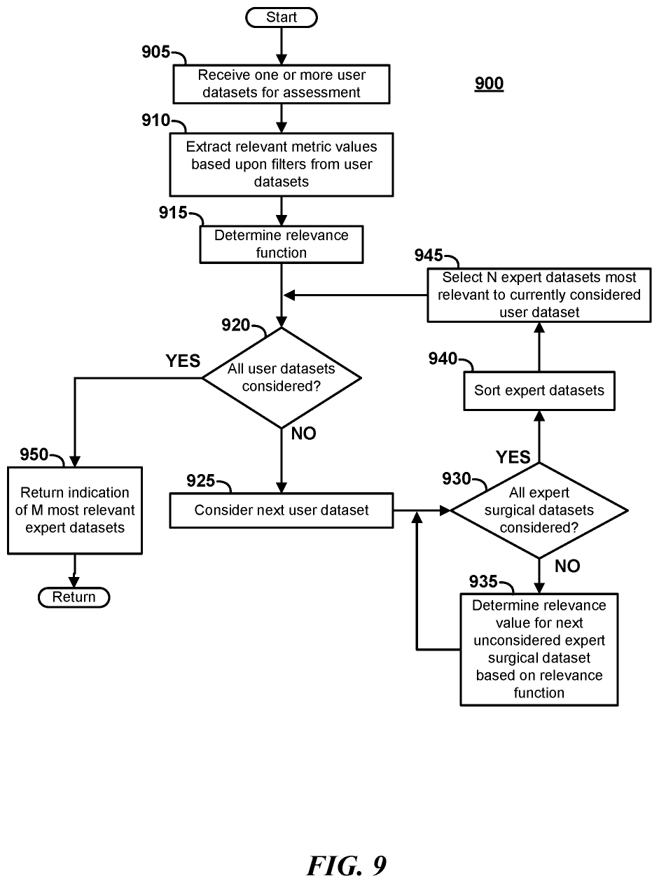

FIG. 9 is a flow diagram illustrating various operations in an example process 900 for selecting recommended surgical datasets based upon one or more user datasets, as may be implemented in some embodiments. The process 900 may be used, e.g., to populate the recommended videos region 835 or to identify expert datasets relevant to a selected user video (such as the video of panel 2005). Given one or more user procedures (e.g., the recent surgical datasets associated with region 810) at block 905, the system may extract the relevant metric values for the procedures based upon the selected dataset filters at block 910. For example, where no task is selected, and only the “total duration” metric is selected, then only the total duration of the procedures may be considered in assessing their relevance. Conversely, if one or more specific metrics and one or more specific tasks are selected, the recommended procedures may be required to have those tasks, and the metric values for those tasks may be used as dimensions by which to assess similarity or dissimilarity for purposes of determining relevance.

For example, each metric may be treated as an independent dimension and the Euclidean distance between metrics values in the user selected procedure and the expert procedure used to identify expert procedures “more distant” from the selected procedure. Where filters established a set of less than all the metrics, then only those metrics appearing in the set may be used in calculating the distance. Similarly, where a filter has been applied for only one or more tasks, then only metrics for those tasks may be considered in the similarity determination. The system may then recommend expert procedures, e.g., in order of decreasing distance, beginning with the most distant procedure (or by increasing distance, depending upon the nature of the contemplated relevance, such as whether similar or dissimilar procedures are desired). While Euclidean distance is referenced herein, one will appreciate variations, as when weighted sums of metrics, principal component vectors, etc. are instead used for assessing surgical procedure similarity/dissimilarity and consequently, relevance to the user surgeon's datasets.

At block 915 the system may determine the appropriate relevance function given the relevance metrics identified at block 910. Again, “relevance” may or may not be the same as “similarity.” For example, if only the metrics “total duration” is considered, then in some situations, the smaller the expert surgery's total duration metric value is relative to a given user surgery, the more relevant that expert's surgery. Thus, the more disparate the relative values, the more “relevant” is the expert surgery in this example. Conversely, a user or the system may filter and specify relevance as being positively correlated with similarity, as for example, where the user wishes to identify surgeries having a specific sequence of tasks performed in a manner similar to the user. Surgeries with additional optional tasks, or which lack any of the specified tasks, or have the tasks in a different order than specified, may be considered more dissimilar and therefore less “relevant” to the user procedure.

Having identified the context factors affecting “relevance,” at blocks 920 and 925 the system may iterate over the user datasets and determine a corresponding relevance value for each expert dataset at blocks 930 and 935, in accordance with the selection at block 915. At block 940, the expert datasets may be ordered based upon their determined values at block 935 and the N most relevant selected at block 945. In some embodiments all N of these datasets may be presented, e.g., in region 835, as when expert surgeries are being identified for only a single user surgery. However, in some embodiments, a different number (i.e., M) from this number of expert surgeries may be returned at block 950. For example, multiple user procedures may appear in region 810, which, at present, are filtered only by their chronology. In this situation, where four user procedures are shown, M may be four and only the most relevant of each of the N identified expert surgeries are returned. Where there are redundancies in the most relevant of each of the N identified expert surgeries, the system may select a second or third most relevant of the N surgeries instead to avoid duplicate return values. The set of ordered datasets identified at block 950 may then be used to populate the video recommendation.

One will appreciate that process 900 is merely exemplary of the considerations contemplated in various embodiments and that the system may perform modified or alternative methods for selecting expert recommended datasets based upon one or more user surgeon datasets.

### Example My Videos Window

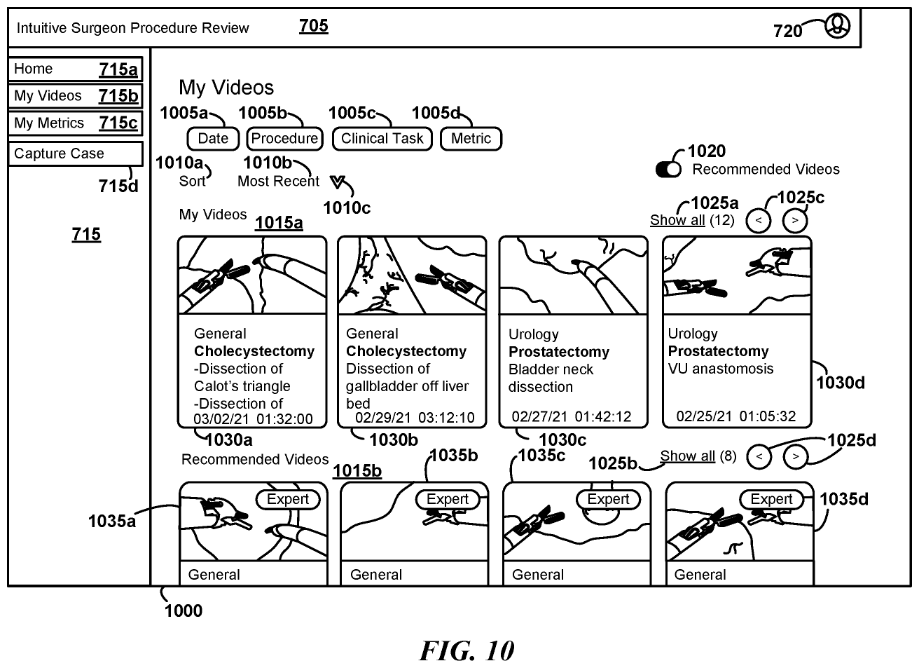

FIG. 10 is a schematic illustration of a computer screen depicting a My Videos window 1000 as may be implemented in some embodiments. Generally, the window 1000 may facilitate the user's sorting through their procedure libraries by procedure type, surgical task(s), objective metrics (DPTs), dates of the surgery, etc. To this end, a plurality of filter selectors 1005a, 1005b, 1005c, and 1005d may be provided. Selecting one of filter selectors 1005a, 1005b, 1005c, and 1005d may present, e.g., an overlaid panel from which the user may select the criteria by which to filter the surgical dataset results appearing in the window 1000. Label 1010a may similarly invite the user to sort the procedures by one of several criteria. For example, after filter selectors 1005a, 1005b, 1005c, and 1005d have identified the subset of all available datasets to present in the window, those results may then be presented in the order specified by the sorting option 1010b. Here, the user has selected the “most recent” sorting option 1010b (i.e., sort chronologically in descending order) via the drop-down indicator 1010c. Changing the sorting option via drop-down 1010c or changing the selected set via filters 1005a, 1005b, 1005c, and 1005d may have the effect of changing the procedure panes presented in the My Videos region 1015a and “Recommended Videos” region 1015b (one will appreciate that the term “video” may be used herein, and in the GUI of the provided figures, to simplify reference to a surgical dataset, which may itself contain sensor and other data in addition to, or, in some cases, in lieu of, camera-captured video data).

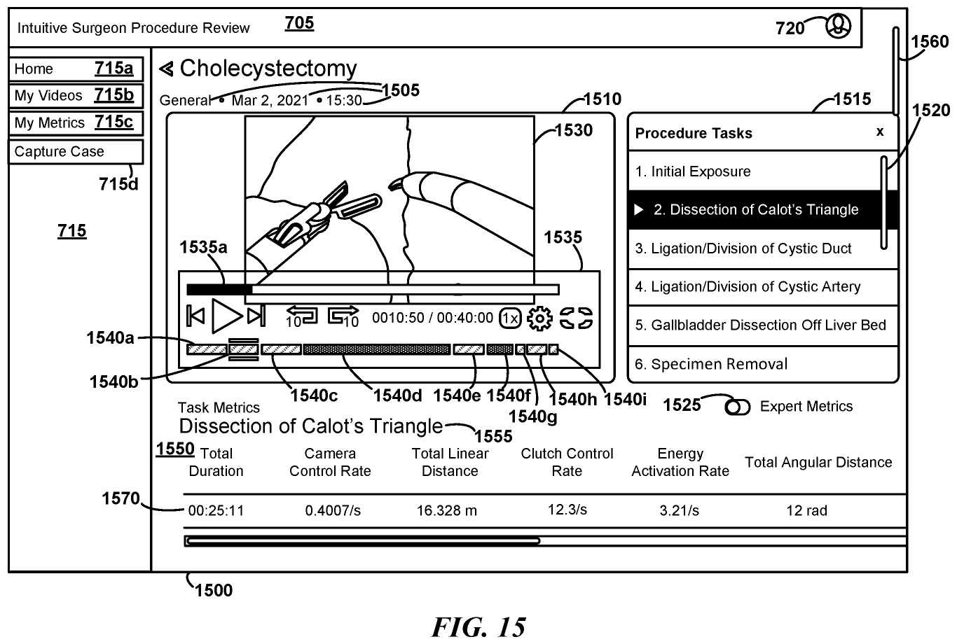

As not all the datasets satisfying the selection criteria may fit onscreen, only a subset may be presented (e.g., the subset of panes 1030a, 1030b, 1030c, and 1030d and the subset of panes 1035a, 1035b, 1035c, and 1035d). The user may iterate between subsets by selecting the left and right selectors 1025c and 1025d for the user's procedures and the recommended procedures respectively. By selecting “Show all” 1025a the user may instead view all of the returned subject procedures (the numerical indication “12” indicating there are 12 total procedures satisfying the filtering criteria specified by filters 1005a, 1005b, 1005c, and 1005d). Similarly, by selecting “Show all” 1025b the user may instead view all of the returned subject procedures (the numerical indication “8” indicating there are 8 total procedures satisfying the filtering criteria specified by filters 1005a, 1005b, 1005c, and 1005d). One will appreciate that where all the procedures are returned, the user may need to scroll down through the window to view them (e.g., where the panes are presented as part of a wrapping “flex-wrap” flexbox CSS element arrangement). Toggling the recommended videos switch 1020 (shown here as being in the “active” position) to an inactive position may remove the recommended videos region 1015b from the window, facilitating a more focused review upon the surgeon's videos in region 1015a. While a single set of filters are applied to both the subject surgeon's videos and the recommended videos in this example, in some embodiments each region may have its own set of filters. Selecting a user or expert video (e.g., left-clicking a mouse upon the pane) may open the corresponding dataset in the Procedure Video window 1500 of FIG. 15 discussed in greater detail herein.

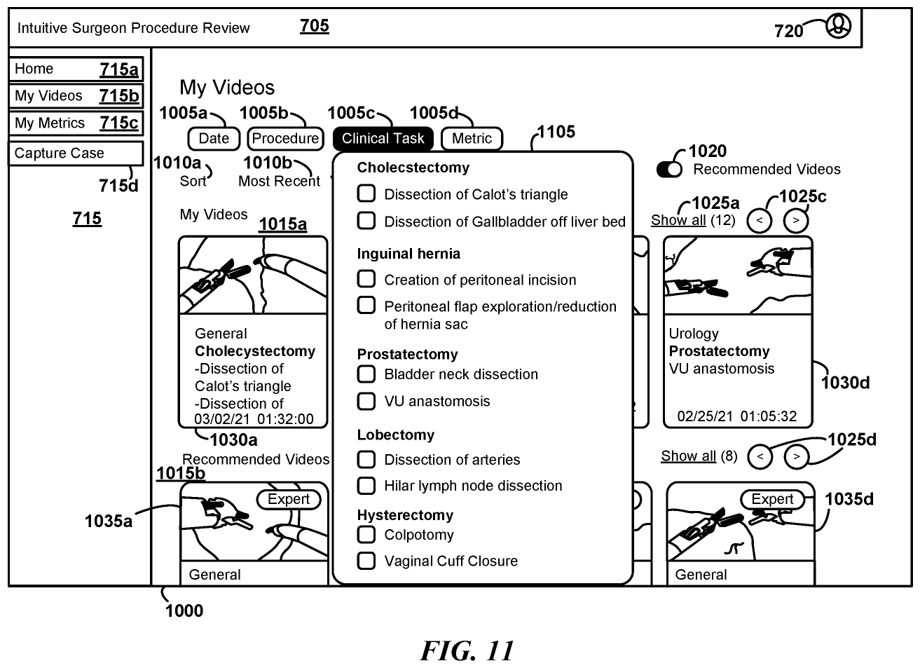

To facilitate clarity of the reader's comprehension, FIG. 11 demonstrates an example activation of the clinical task filter 1005c (e.g., clicking upon it with a mouse), presenting overlaid filter drop-down pane 1105, as may be implemented in some embodiments. Specifically, by selecting one or more of the checkbox buttons appearing next to each task (e.g., “Dissection of Calot's Triangle”) for the respective procedures (e.g., “Cholecystectomy”), only datasets which include data for that task, for that procedure, will be included in the results (though, in the absence of any selected checkboxes, all the available videos may be shown in this example). In some embodiments, the video preview in each of the remaining panes may be offset to the beginning frame of the earliest instance of the filtered tasks, thereby facilitating the user's quick review of the datasets for the tasks in question. In some embodiments, where multiple tasks are selected, the video preview may alter between frames of portions of the video corresponding to the respectively selected tasks (e.g., if two tasks are selected, a slowly transitioning slideshow may be presented of frames corresponding to the first task and of frames corresponding to the second task). While tasks are shown for multiple procedure types in this example merely to facilitate understanding, one will appreciate that in some embodiments pane 1105 will show only tasks for the one or more procedures selected via filter 1005b (which may also present a pane of checkbox selections for procedure types). Similarly, a specialty filter may be provided in some embodiments, facilitating filtering by specialties, then procedures, then tasks, and then metrics (such a specialty selection may be useful where the surgeon practices more than one specialty, or where the user is reviewing multiple surgeons at once, various of the surgeons operating in different specialties).

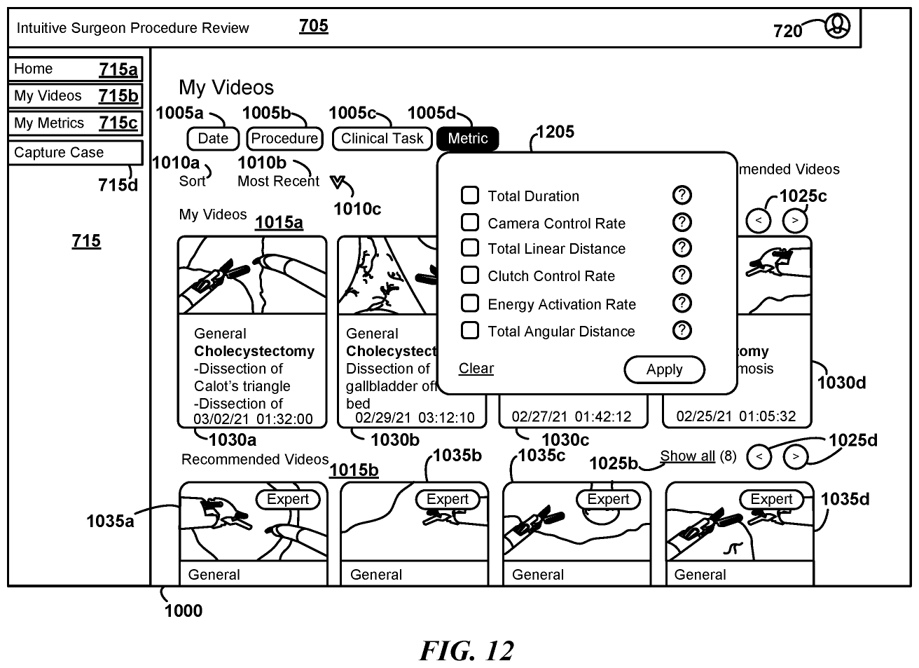

Similar to FIG. 11, FIG. 12 depicts the overlay of metric filter drop-down 1205 after the user has selected the OPI metric filter 1005d, as may occur in some embodiments. Selection of one or more for the checkboxes appearing in filter drop-down overly 1205 may limit the displayed datasets to those surgical datasets where the metrics appear (e.g., “Camera Control Rate”) in the surgery as a whole, or in a previously selected task from filter 1005c. For example, not all tasks may employ energy activation and so tasks without activation may not have values for the energy activation rate OPI metric. In some embodiments, the retained preview panes may be arranged in descending order in accordance with the duration during which the metric applies to the tasks in the procedure. As the user may not be familiar with the meaning of each OPI metric, selection of the question mark icon beside each metric in filter drop-down overly 1205 will present an overlaid explanation of the metric (e.g., like those appearing in the tables of FIGS. 23-26).

### Example Metrics Analysis Window

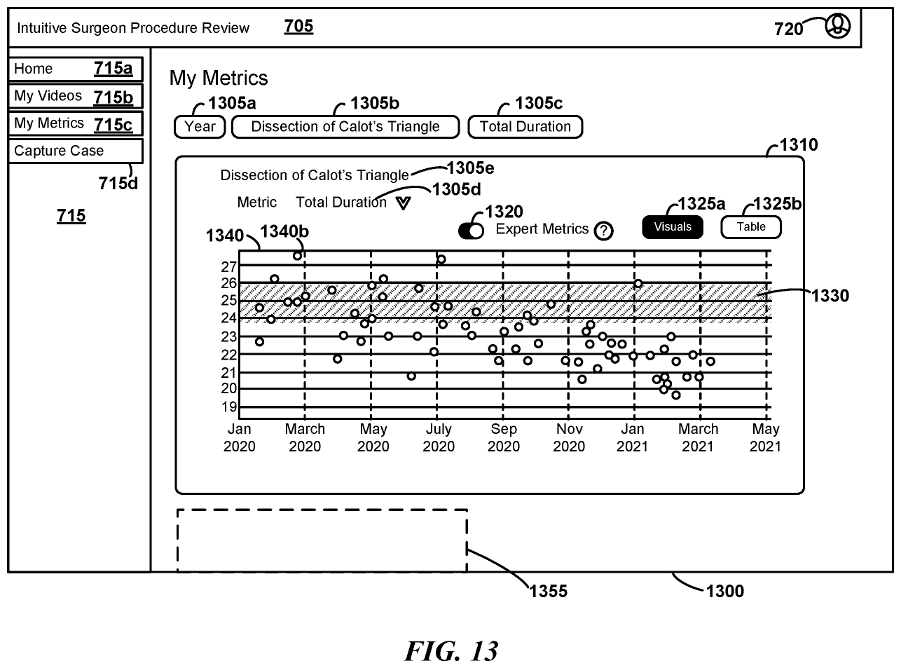

FIG. 13 is a schematic illustration of a computer screen depicting a My Metrics window 1300 with a scatter plot metric map presentation, as may be implemented in some embodiments. In this example, the system may present a temporal selection 1305a, a task selection 1305b (the user has selected a “Dissection of Calot's Triangle” task, e.g., via a drop-down presented by clicking task selection 1305b), and an OPI metric selection 1305c (here, the “total duration” OPI metric has been selected). As previously discussed, additional filters (e.g., for procedure and specialty) may also be provided in some embodiments. These selections may serve as filters for choosing datasets depicting the task of interest and arrangement in accordance with a given metric value (e.g., here the scatter plot axis is chosen to be the metric “total duration”). For example, temporal selection 1305a may restrict the selected surgeries to a first set, task selection 1305b may then select a subset of that first set, and then metric selection 1305c may select a final subset of that subset as the set of datasets appearing in scatter plot 1340 having the selected metric value in the task (or the metric selection 1305c may simply serve to identify the metric value used for sorting, rather than serve to further limit the selected set). In some embodiments, more than the single task selection (“Dissection of Calot's Triangle”) or a single metric selection may be provided, as when the user selects for more than one task or metric at a time. Filters for procedures and procedure specialties may also be provided, as described elsewhere herein. Similarly, filters may be configured to select for metrics based upon tasks (e.g., making it possible to further filter based only upon metrics, or metric value ranges, appearing in the selected tasks). Initially, prior to filtering, the set of procedures appearing in scatter plot 1340 may be a “default set,” such as, e.g., all of the stored user procedures, or all of the procedures occurring in the past year. In some embodiments, an additional metric filter selector may be provided via drop-down 1305d, so that the user may quickly adjust the plot 1340 (in some embodiments, changing drop-down 1305d may likewise change filter 1305c and vice versa).

The selected metric may determine the Y-axis of the scatter plot 1340 appearing in region 1310. Here, the “total duration” metric of the “Dissection of Calot's Triangle” task has been selected and so the duration in minutes of that task is presented along the Y-axis (as indicated, the range of the Y-axis may also be chosen based upon the minimum and maximum values of the metric in the filtered datasets). A task selection label 1305e may help remind the reader of the presently filtered task. Each point in the scatter plot 1340 corresponds to a dataset acquired during one of the subject surgeon's surgeries and each point's position along the Y-axis corresponds to the total duration of the “Dissection of Calot's Triangle” task appearing therein. Thus, the scatter plot 1340 forms a “metric map,” mapping one or more metric values to graphical icon representations (points in a scatter plot, rows in a table, etc.) of surgical datasets. For example, the point 1340b corresponds to a surgery performed by the subject surgeon in late February 2020, during which the “total duration” metric value for the “Dissection of Calot's Triangle” task was almost 28 minutes (one will appreciate that these numbers are chosen merely to facilitate understanding and that the actual “Dissection of Calot's Triangle” task, in the real world, may not typically correspond to such durations). In this example, clicking, or otherwise selecting the point 1340b will present the corresponding dataset in a Procedure View window, e.g., as discussed herein with respect to FIG. 15. In some embodiments, hovering over a data point, such as point 1340b, will present case metadata (e.g., as an overlay display) along with the exact metric value. In some embodiments, the user may be able to zoom to portions of the plot 1340, traverse the plot 1340 via sliders, and scale the scatter plot 1340 to facilitate quick consideration of the points' relative values.

Activation of the expert metrics toggle switch 1320 may present a range 1330, e.g., a colored region within the scatter plot, indicating metric values corresponding to a number of expert surgeons (e.g., the range of values for the top 75%, middle 50%, all the experts, the range found by one standard deviation above and one standard deviation below the average or median expert metric value, etc.). One can see from that example scatter plot that the subject surgeon's time for performing the “Dissection of Calot's Triangle” task has generally decreased over several months to the point that it is even well below that of many experts by March 2021.

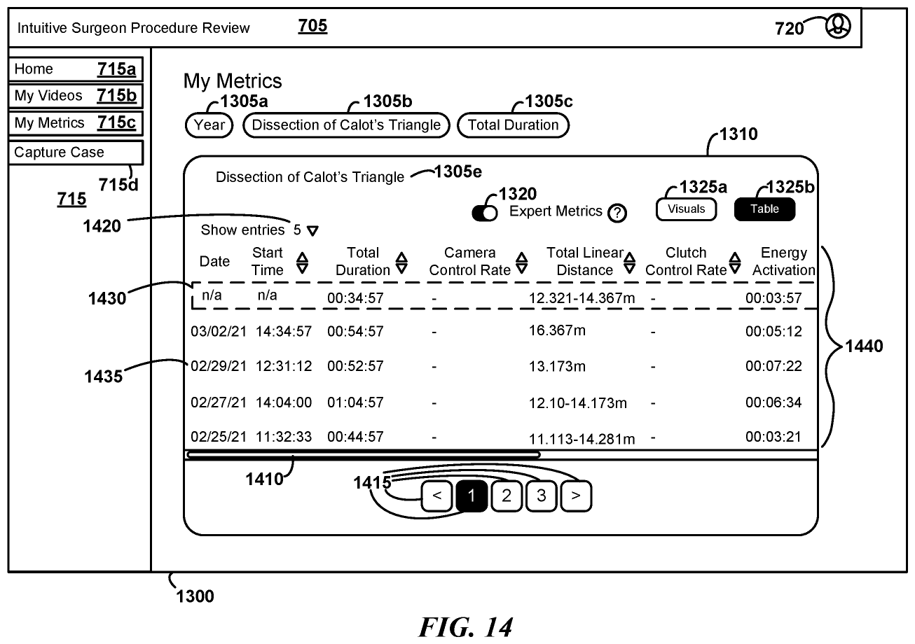

While selecting the “Visuals” button 1325a presents the depicted scatter plot, as in this example, instead selecting the “Table” button 1325b may present the data in a tabular format, e.g., as shown in FIG. 14. In FIG. 14, rather than represent individual subject surgeon datasets with points in a scatter plot, each surgery dataset is represented as a row in a table. Each row may depict a plurality of metric values (rather than just the “total duration”) for the “Dissection of Calot's Triangle” task in a given surgery (indeed, scroll bar 1410 indicates that additional columns of metric values may be shown if scrolled to the right). Similar to how selecting a point in the scatter plot 1340 will present the corresponding surgical dataset in the Procedure View window 1500, selecting a row in the table of FIG. 14 will likewise present the surgical dataset corresponding to that row in the Procedure View window 1500. Thus, the table also serves as a metric map. As there may not be enough room to show all the table's rows at once, paging controls 1415 may facilitate selection of subsets of the rows. In this example, the user has chosen to show five rows at a time, per the selection drop-down 1420. Here, rather than highlight a portion of the scatter plot, activation of the expert metrics toggle switch 1320 may present an additional row 1430 on each page of rows, the row 1430 indicating the average expert value or range of expert values for each of the metrics. Ranges, averages, distributions, etc. across experts may be more useful to the user's review than presenting the individual values for a single expert surgery.

Again, as each of scatter plot 1340 and the table 1440 of FIG. 14 (as well as the plot 1720 to be discussed herein) relate metric values to surgical datasets, they are each referred to herein as “metric maps.” One will appreciate a variety of ways to visually depict such metric maps other than by scatter plots and tables, including, e.g., histograms, density maps, bubble plots, radial plots associating, e.g., corners to metrics, dendrograms, circular packings, etc., in each case relating a plurality of surgical datasets to one or more corresponding OPI metric values and facilitating the dataset's deeper consideration by selecting an iconic representation of that relation. One will appreciate that the numerical values appearing in table 1440 are provided to facilitate understanding and may not necessarily correspond to values appearing in a real-world implementation.

### Example Procedure View Window

As discussed, multiple transition paths may bring the user to a Procedure View window 1500 as shown in FIG. 15. For example, selection of one of panes 815, 820, 825, 830 or one of panes 1030a, 1030b, 1030c, 1030d, or selection of a point on the scatter plot 1340, or selection of a row, e.g., row 1435, in the table of FIG. 14, etc. may populate Procedure View window 1500 with the selected surgical procedure data. While some of these selections may be made without regard to a specific task or metric (e.g., the panes 815 and 1030a), as discussed, some selections, such as through a scatter plot point arranged in accordance with a specific task's metric, may inform the representation in window 1500 (e.g., advancing the playback to the first frame of a filtered task occurring in the surgery, highlighting a filter metric value, etc.).

Generally, the window 1500 may provide video playback functionality of the selected surgical case, via a playback interface 1510. Labels 1505 may indicate the selected procedure's specialty (“General”), date (“Mar. 2, 2021”) and time of the procedure's performance (“15:30”). Interface 1510 may include a playback region 1530 depicting video, such as endoscopic video, from the surgery and corresponding controls 1535 (e.g., play, rewind, fast forward, change playback speed, etc.). A progress bar 1535a may indicate the position of the currently depicted frame in the playback. Below the playback controls are shown a series of rectangles 1540a, 1540b, 1540c, 1540d, 1540e, 1540f, 1540g, 1540h, 1540i. The rectangles 1540a-i may correspond to tasks performed during the surgery and may also be represented by entries in the procedure task pane 1515, with the currently depicted task being highlighted, bolded, or otherwise identified (as is the second task in this example). Thus, each of procedure task pane 1515 and rectangles 1540a-l are task indication interfaces, facilitating selection of a specific task in the playback. Below playback interface 1510 and pane 1515 is a task-metrics region 1550 depicting OPI values relevant to the currently depicted task. Each of the tasks pane 1515, rectangles 1540a-i and progress bar 1535a may correspond to one another and be updated so as to retain that correspondence as the playback advances (as in this example, rectangles 1540a-l may cumulatively be approximately the same length as the full range of progress bar 1535a to visually emphasize the correspondence). Metrics appearing for the task in the row 1570 of region 1550 below the playback may likewise be adjusted as playback advances. Accordingly, in the currently depicted moment, the playback region 1530 depicts a frame from the surgery during the second task (indicated by the progress bar's 1535a reaching the highlighted rectangle 1540b, and the highlighting of the second task “Dissection of Calot's Triangle” in the task pane 1515). The metrics in row 1570 of the region 1550 likewise correspond to this task. As there are nine tasks, but only six are visible at a time in task pane 1515, a scroll bar 1520 may be provided so that the user can scroll to the non-visible tasks. Just as clicking on a portion of the progress bar 1535a will move playback 1530 to the corresponding time (and update the task indications in the rectangles 1540a-i, pane 1515, and metrics in the table below), clicking on either one of the task rectangles 1540a-i or upon one of the tasks in task pane 1515 may move the progress bar 1535a and playback 1530 to a time corresponding to the beginning of the selected task, as well as update the OPI metrics appearing in the row 1570 of region 1550. One will appreciate that in some embodiments “null tasks” may be present during periods wherein no task is being performed.

A label 1555 may reiterate the current task to the user (and may likewise be adjusted as playback advances). Here, as the displayed task is “Dissection of Calot's triangle,” the same appears in the label 1555. One will recognize that the table of OPI values shown in the region 1550 may be generally the same as that shown in the corresponding row of FIG. 14 for the given task. Similarly, selection of the expert metrics toggle 1525 (shown here in a deactivated state) may present an additional row depicting expert OPI values for the task (analogous, e.g., to the presentation in row 1430).

As will be discussed in greater detail below, in some embodiments, functionality below the portion of the procedure window displayed in FIG. 15 may facilitate rapid iteration between surgical procedure datasets. This is reflected in FIG. 15 by the presence of window-level scroll bar 1560 near the top of the window (i.e., indicating that the user is viewing a top portion of the page and that more features are available below).

### Example Procedure View Window—Expert Mirroring

Some embodiments may implement a variation of Procedure View window 1500, as shown in window 1600, presenting an additional video playback interface 1610 depicting an expert video exemplary of the depicted procedure or task. Similar to the expert values row 1430, the system may also provide row 1605 showing expert metric values (including ranges, distributions, etc.) for the current task. In some embodiments, row 1605 instead depicts the current metrics for the expert appearing in playback interface 1610. As will be discussed in greater detail herein, in some embodiments, interface 1610 and row 1605 may be always provided in the Procedure View by default, whereas in some embodiments they are only provided following user selection of a recommended video, whereas in still other embodiments, they may both appear, or only one may appear, following activation of the expert metrics toggle 1525.

In some embodiments, the expert video shown in video playback interface 1610 may be the same for all the tasks in the surgery shown in playback interface 1510. However, in some embodiments, different expert videos may be presented in video playback interface 1610 for different tasks as the most “exemplary” performance may not appear in the same video (in some embodiments, the user may select whether to permit such transitions or to retain the same expert video throughout the entire playback). In some embodiments, when a task has been selected, each of playback interface 1510 and playback interface 1610 may play at normal speed at the first frame of the selected task and the metrics appearing in regions 1605 (e.g., the depicted expert's metrics or the consolidated metrics of experts) and region 1570 (the user's metrics) may be updated to reflect the values for the newly selected task. This may allow the user to assess their relative performance and compare individual metrics between the surgeries. For example, the user may periodically pause one or both of the videos and compare individual metric values, such as camera control rate, forceps motion, etc., iteratively playing portions of the videos so as to get a feel for the comparative performance.

Accordingly, it may sometimes benefit the user to avoid focusing upon a specific metric or portion of a video to the exclusion of a more holistic consideration of other portions of the surgeries. Thus, rather than present metric values for only the presently depicted moments in the respective videos, in some embodiments, an average or cumulative value for the whole task may be shown. Similarly, in the expert row 1605 ranges of values across all or some of the experts may be shown alongside, or in lieu of, the metrics for the particularly depicted expert video. In this manner, the user may be able to assess how their performance (specifically the metric values in row 1570) compares to the distribution of expert values. Plots of the expert distributions may be similarly provided so as to provide an intuitive feel for the user's relative performance.

While both playback interface 1510 and playback interface 1610 may be played at their normal speeds in some embodiments, in some embodiments, one or both, of their speeds may be adjusted so that the user can observe their relative progress. For example, where the expert completes a task in half the time it took the subject surgeon, the surgeon's playback may be accelerated to match the duration of the expert (in some embodiments, the metrics rows values may likewise update at different rates). In this manner, the subject surgeon can observe how much faster they would need to perform their chosen operations so as to achieve the expert's duration.

### Example Procedure View Window—Quick Access Metric Map

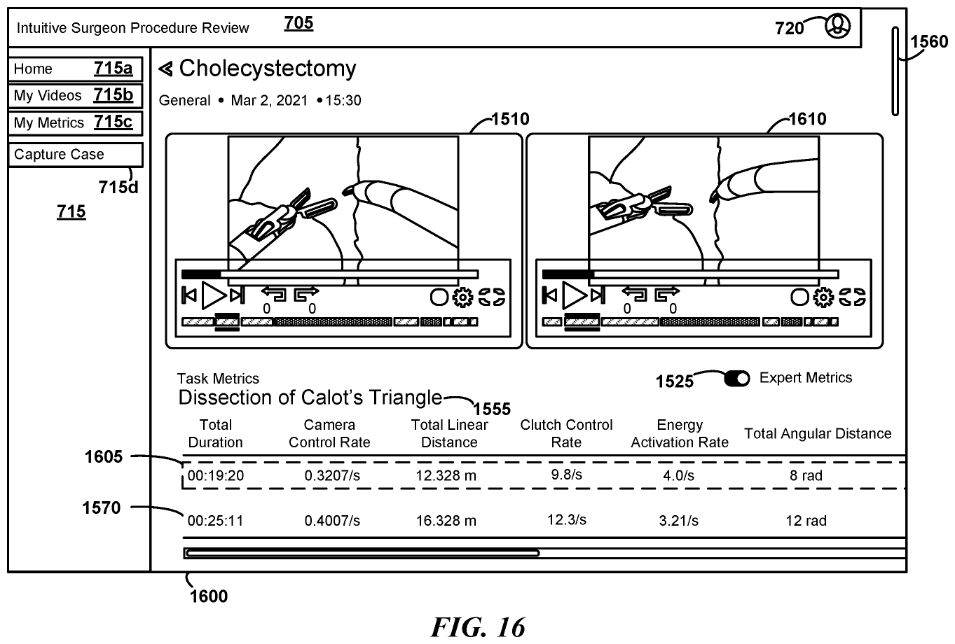

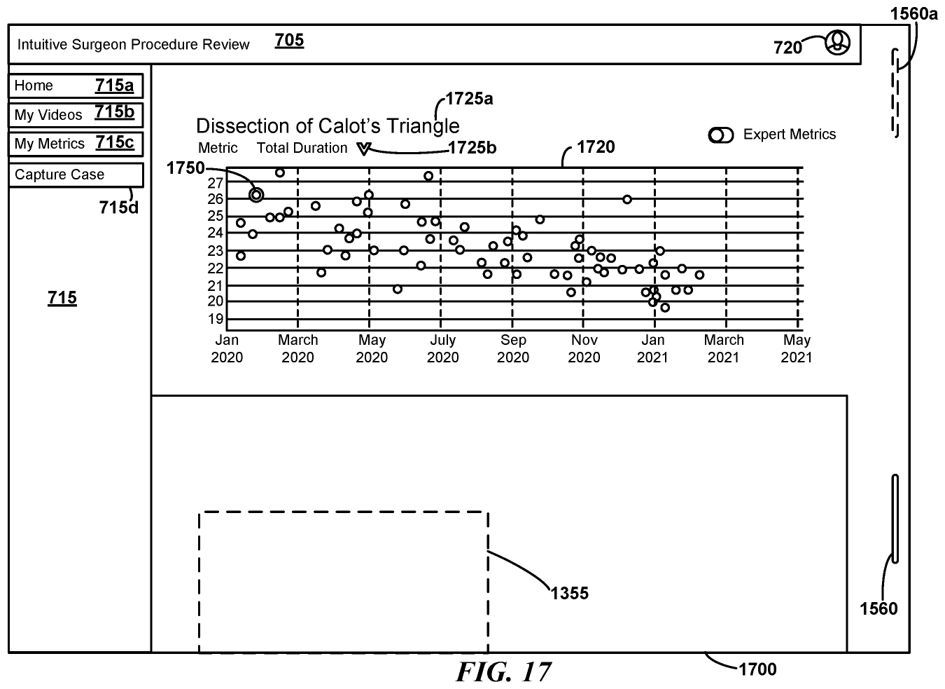

Some embodiments seek to facilitate rapid, iterative review by the user of multiple surgical procedures (e.g., as when returning to the Procedure View window via transition 615). To this end, FIG. 17 depicts a schematic computer screen layout showing a portion of Procedure View window 1700 including a scatter plot 1720 analogous to the plot 1340 on the My Metrics window 1300 (one may display a corresponding expert range 1330 in scatter plot 1720 by selecting the expert metrics toggle 1775). Scatter plot 1720 (or a table, bubble plot, dendrogram, etc.) is referred to as a “quick access” metric map, since it appears within the Procedure View window, facilitating quick presentation of a new procedure in an updated Procedure View window. In this example, the user may scroll down from the view depicted in either FIG. 15 or FIG. 16 to reveal the portion depicted in FIG. 17 (navigation bar region 715 and top bar region 705 remaining fixed). Accordingly, window-level scroll bar 1560 is shown here at a much lower position in the window relative to its previous position 1560a in FIGS. 15 and 16. In some embodiments, the quick access metric map may instead by presented as an overlay, dropdown panel, etc. Similar to My Metrics window 1300, a label 1725a may indicate the presently selected/viewed task and a drop-down 1725b may indicate the OPI metric of that task used for generating the plot 1720.

Like the scatter plot 1340, clicking on any of the points in the scatter plot 1720 will cause the system to present a Procedure View window, e.g., the window 1500 or the window 1600 populated with the surgical data corresponding to that point. Unlike the plot 1340, however, the surgery presently loaded by the Procedure View window may be highlighted, e.g., with a different color, border, a specific annotation, such as a box pointing to the highlighted dot with the text “selected case”, etc. Here, e.g., the point 1750 associated with the currently selected procedure is highlighted. Note that as the “Dissection of Calot's Triangle” task is presently selected, as indicated by label 1725a, the system may advance the playback (e.g., interface 1510 or 1610), selected task (e.g., in one of rectangles 1540a-l and procedure task pane 1515), and task metrics (e.g., highlighting columns of row 1570 or row 1605), to the frame where that task appears for the newly selected procedure after transitioning to the updated Procedure View window. In this manner, quick and iterative review of a specific task across procedures may be made available to the analyst, avoiding disruption to the analyst's cognitive flow.

### Example Procedure View Window Relative Component Placement

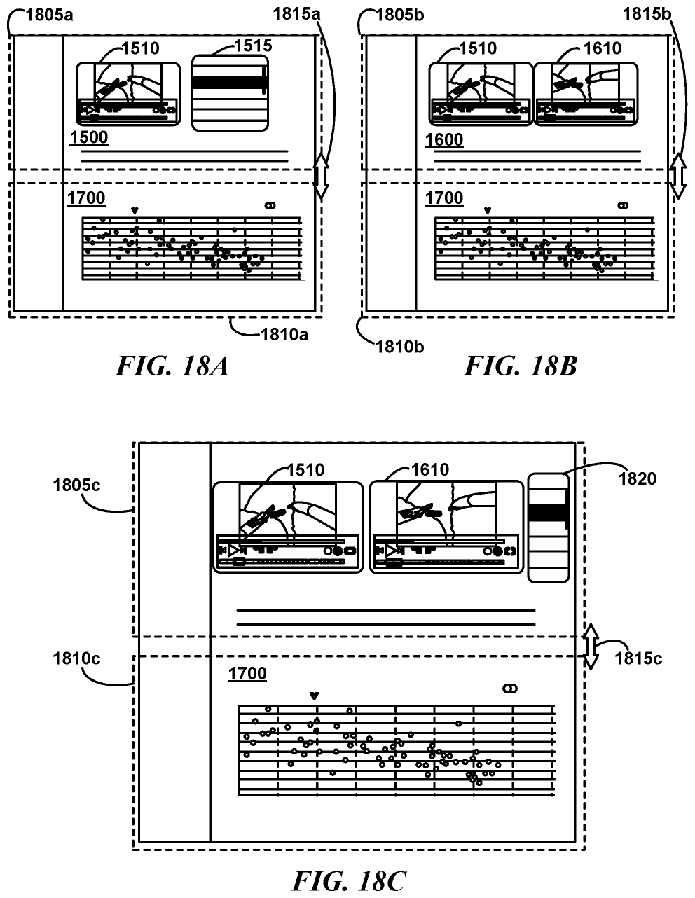

For clarity in the reader's comprehension, FIGS. 18A-C depict extended schematic computer screen layouts illustrating example relative positions of the video portion of the Procedure View window of FIGS. 15, 16 and the portion of Procedure View window depicted in FIG. 17, as may be implemented in some embodiments. Specifically, FIG. 18A illustrates the relation between the view for window 1500 and window 1700 where the views comprise portions of the same HTML webpage and may be viewed by scrolling 1815a between them via window-level scroll bar 1560. This behavior may follow from, e.g., the browser's window dimensions accommodating only one of portions 1805a and 1810a of the page at one time. Again, in this example the positions of top bar region 705 and navigation bar region 715 may not change in each of window 1500 and window 1700 as top bar region 705 and navigation bar region 715 are “fixed.”

FIG. 18B similarly depicts the relation between window 1600 and window 1700. Again, in this example, both windows appear as portions of the same HTML webpage and may be viewed by scrolling 1815b between them via window-level scroll bar 1560. This behavior may follow from, e.g., the user's browser being able to view only one of portions 1805b and 1810b of the page at one time. Similarly, the positions of top bar region 705 and navigation bar region 715 may not change in each of window 1600 and window 1700 as they are “fixed.”

One will appreciate that some embodiments may combine features of FIGS. 18A and 18B to facilitate user navigation. For example, while some users may be comfortable navigating between tasks using rectangles 1540a-i within window 1600, some users may prefer to use task panes analogous to procedure task pane 1515. To this end, some embodiments may also supply one or more task panes, such as task pane 1820, to facilitate navigation between one or both video playbacks. Accordingly, selecting a task in the task pane 1820 may adjust each of the playback interfaces 1510 and 1610 (as well as corresponding task rectangles and OPI metrics table) to the newly selected task. In some embodiments, each playback interface may be associated with its own task pane, selections in a task pane precipitating playback adjustments in only the corresponding playback interface, metrics row, etc. As discussed with respect to FIGS. 18A and 18B, the user may scroll 1815c between each of the portions 1805c and 1810c of the page.

### Example Procedure View Drill-Down Process

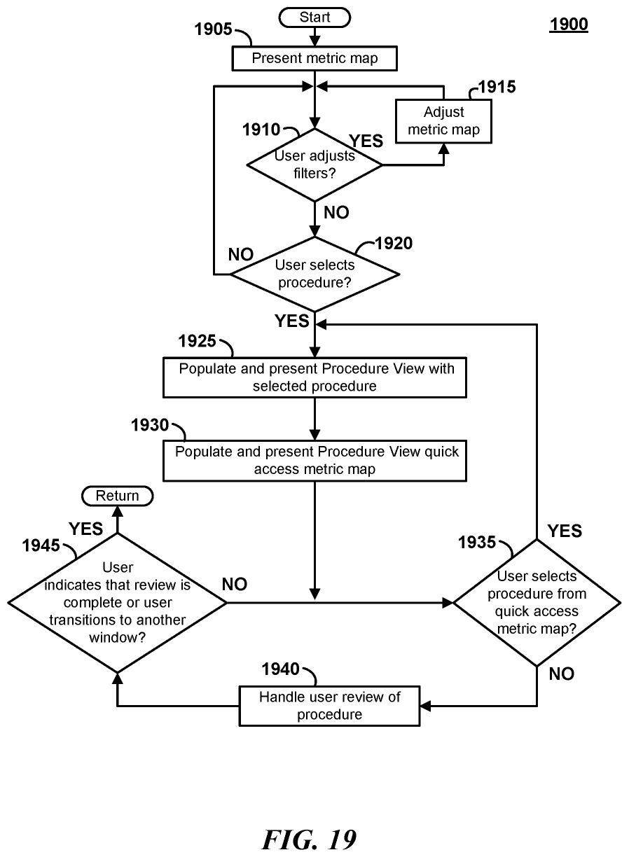

FIG. 19 is a flow diagram illustrating various operations in an example “procedure-view drill-down” process 1900 for user procedures, as may be implemented in some embodiments. Specifically, at block 1905, the system may present the user with a metric map, e.g., the scatter plot 1340 map appearing in the My Metrics window 1300, or the metric map table 1440. Where the user adjusts one or more filters at block 1910, e.g., one of filters 1305a, 1305b, 1305c, the map may be adjusted at block 1915 (e.g., new sets of surgical procedures and their metrics presented in the table, a new range or plot of points on the scatter plot for a new set of surgeries, etc.).

Where the user selects a procedure from the metric map at block 1920 (selecting a row in table 1440, selecting a point in scatter plot 1340, etc.), the system may transition to the Procedure View window 1500 and present the selected procedure, e.g., as shown in FIG. 15, at block 1925. Similarly, at block 1930, a quick access metric map may be presented, e.g., such as the scatter plot 1720 (or an equivalent table). The quick access map may already focus upon those surgical procedures specified by the filters at block 1910. Until the user selects a new procedure from the quick access map at block 1935, the system may handle the user's review of the currently selected procedure at block 1940 (e.g., playback operations, metrics values display, etc.). The user may choose to close the program, or travel to another page, as indicated at block 1945, thereby concluding the quick access iterative consideration of procedures. One will appreciate, however, per FIG. 6 (particularly transition 605i) that the user may be again travelling to the My Metrics window, which may initiate a new review at block 1905. Where procedures continue to be selected via the quick access map at block 1935, however, the system will continue to populate and present Procedure View window 1500 at block 1925.

Example Procedure View Metric Map with Intermediate Selection

While selecting an item in a metric map (whether the metric map of the My Metrics window or a quick access metric map within the Procedure View window) may immediately take the user to a Procedure View window depicting the selected procedures in some embodiments, some embodiments may first present a selection confirmation panel before effecting the transition. Such a panel may be particularly useful in embodiments presenting or facilitating presentation of interface 1610, as interposing the selection confirmation may facilitate an appropriate choice of recommended expert video as well as help direct the user procedure and task selection for review. Interposing the expert video selection in this manner may provide higher impact results earlier in the user's review, as the user is not obligated to fully transition to the Procedure View before considering the appropriateness of the selected user surgical dataset and recommended dataset. Involving the user in the expert dataset selection may help fine-tune the expert dataset recommendation in accordance with the user's expressed focus.

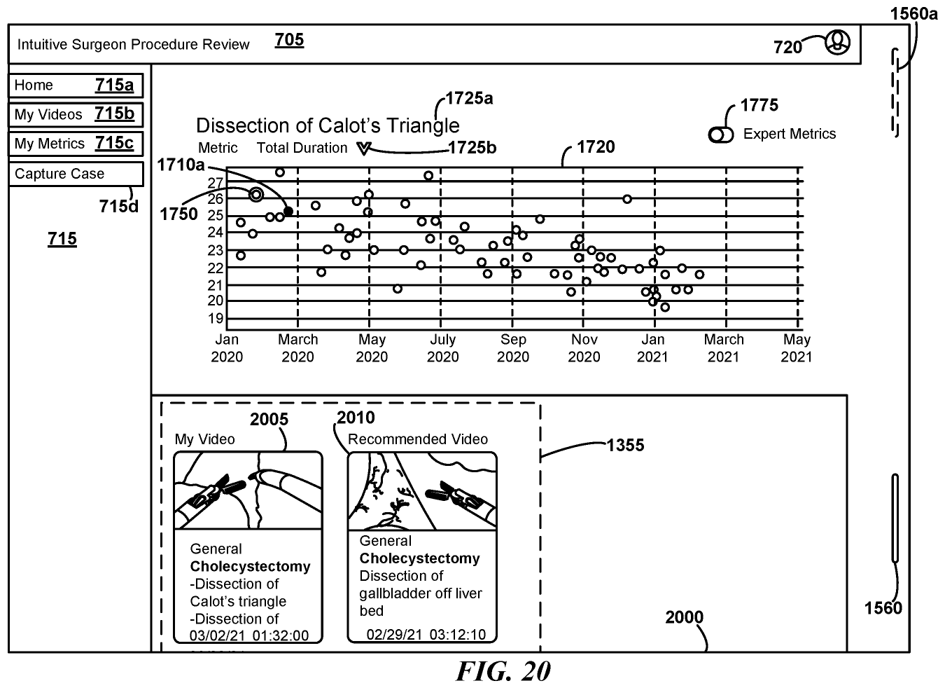

Specifically, FIG. 20 is a schematic computer screen layout depicting a portion of Procedure View window 2000 with a scatter plot quick-access metric map and intermediate selection panels, as may be implemented in some embodiments. As indicated, the window 2000 may include many or the same features as was described with respect to FIG. 17, with the modifications and addition described herein.

Particularly, in each of windows 1300 and 1700 a region 1355 may be reserved, in some embodiments, for presenting intermediate panels, e.g., after selecting a surgical procedure and before transitioning to the updated Procedure View window. In contrast, in some embodiments, the region 1355 may not appear in the window and the intermediate panels may be presented in a pop-up pane, overlaid panel, slide-down panel, etc. In this example, panels 2005 and 2010 appear in the region 1355 following selection of the point 1710a (here shown as highlighted via color, opacity, etc., to confirm its selection) in lieu of an immediate transition to the new Procedure View window. As before, a highlight indicates that the surgical procedure associated with point 1750 is presently displayed in the Procedure View window. Highlighting each of points 1750 and 1710a in this manner may help emphasize their relative chronological and metric values to the user. As indicated in FIGS. 13 and 17, region 1355 may appear in both a metric map of the My Metrics window and in a metric map (e.g., a quick access metric map) of the “Procedure View” window. In embodiments where the intermediate panels are presented prior to transitioning to a “Procedure View” window populated with the new surgical data, these regions 1355 may likewise be populated with panels such as panels 2005 and 2010.

Panel 2005 may present confirmation of the user's procedure selection (i.e., the dataset corresponding to point 1710a) and high-level information regarding the surgical dataset, e.g., the same information as in the region 1015a of the My Videos window 1000. This high-level information may help the user appreciate whether the selected dataset contains relevant/desired tasks, skills, metrics, etc. for their review. Selecting the panel 2005 (e.g. clicking upon it) may cause the transition to the new Procedure View window to proceed. Similarly, analogous to the presentation of videos in region 835 or 1015b using, e.g., the process 900, the intermediate panels may include one or more recommended videos in panel 2010. In some embodiments, the most relevant of the recommended videos may be used as the default video loaded into interface 1610 and row 1605. One will appreciate that in some embodiments the system may present only panel 2005, rather than both panel 2005 and panel 2010.

Example Procedure View Metric Map with Intermediate Selection—Example Drill-Down Process

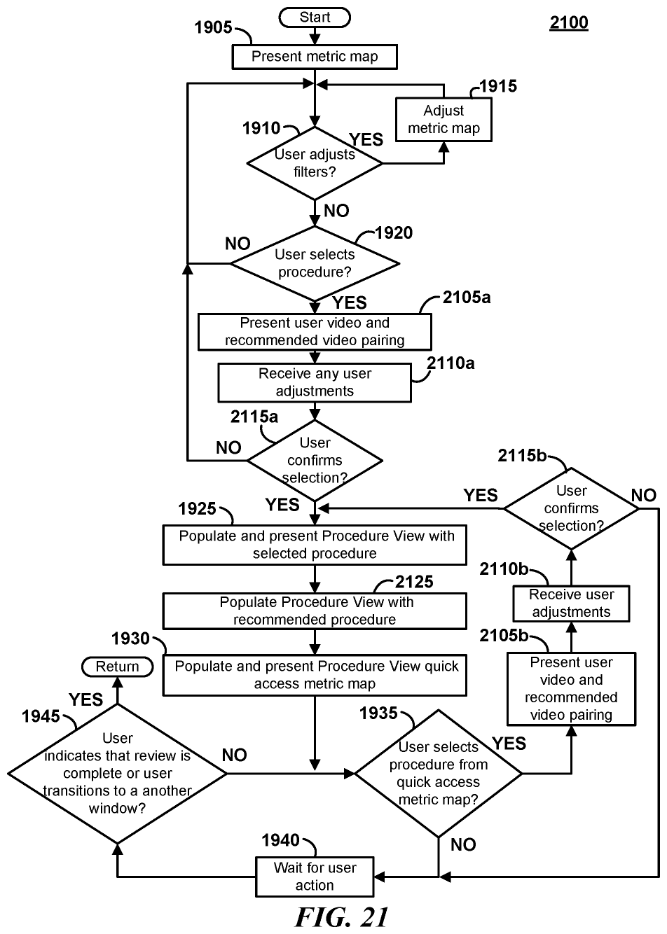

FIG. 21 is a flow diagram illustrating various operations in an example “procedure-view drill-down” process 2100 for both user and recommended procedures, as may be implemented in some embodiments. As indicated, the process 2100 may include many of the same operations discussed with respect to process 1900 (such as presenting a metric map at block 1905 in, e.g., a My Metrics window, presenting a quick access map at block 1930, etc.). However, process 2100 additionally includes operations involving the presentation and operation of intermediate panels, shown in the groups of actions at blocks 2105a, 2110a, 2115a when transitioning from, e.g., the My Metrics window via the scatter plot 1340 metric map and groups of actions at blocks 2105b, 2110b, 2115b when transitioning from, e.g., the quick access scatter plot 1720 metric map of the Procedure View. Specifically, after receiving a user surgical procedure selection at block 1920 or at block 1935, the system may now instead present the intermediate panels (e.g., panels 2005 and 2010) at blocks 2105a, 2105b, respectively. In some embodiments, the system may receive user adjustments at blocks 2110a, 2110b, e.g., where the user selects a different recommended video from a list in panel 2010. As discussed, one will appreciate that the user may elect not to make any adjustment at blocks 2110a, 2110b, accepting the default recommended procedure. However, where the user selects a different recommended procedure at block 2110, the default value choice may be replaced with the new selection, as when the user, e.g., disagrees with the system's selection made based, e.g., on the process 900 (in some embodiments, the process 900 may consider such adjustments in the relevance function selected at block 915, disfavoring recommendations eschewed by the user that might otherwise have been given a more favorable relevance ranking). Once the user confirms their selection (e.g., clicking panel 2005) at blocks 2115a, 2115b the system may proceed, as indicated, to the new Procedure View window populated with the selected datasets. In some embodiments, the user may be able to explicitly decline to proceed at blocks 2115a, 2115b, causing the intermediate panel to disappear, before returning to the My Metrics or Procedure View window for review. Once the user proceeds to the Procedure View window, in addition to the presentation at blocks 1925 and 1930, at block 2125, the system may populate the window with the selected recommended expert procedure, e.g., populating row 1605 and interface 1610 (as well as, e.g., advancing the values to the corresponding selected task).

### Example Procedure View Drill-Down Configuration Process

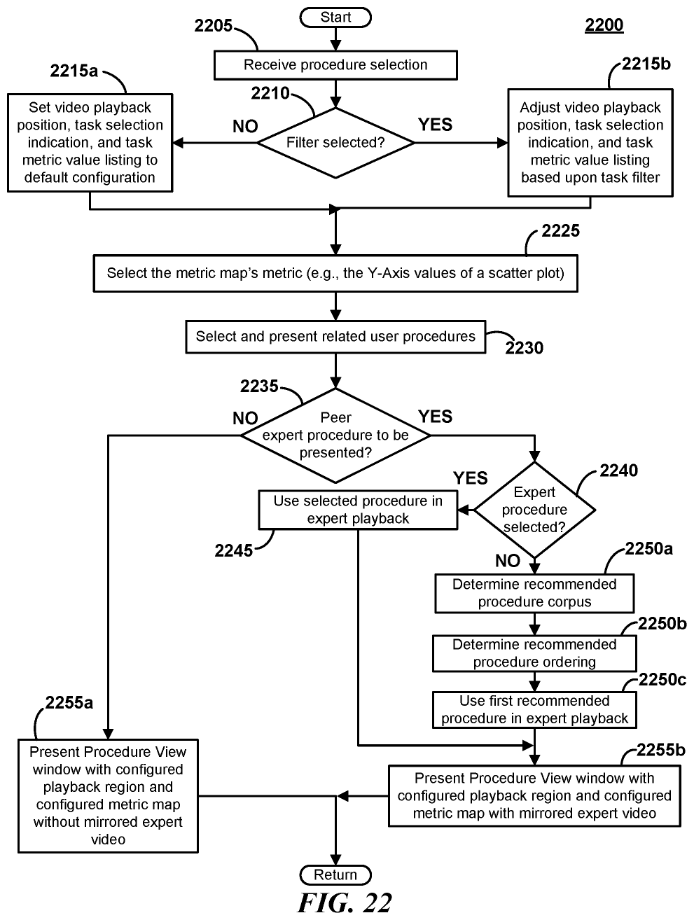

FIG. 22 is a flow diagram illustrating various operations in a “procedure-view drill-down” configuration process 2200 as may be implemented in some embodiments. The “procedure-view drill-down” configuration process 2200 may allow the user to approach the Procedure View window and the information therein in an efficient and intuitive manner, configuring and prepopulating the window in accordance with the user's current state of review. Accordingly, the configuration process 2200 may occur, e.g., in conjunction with blocks 1925, 1930, 2125.

At block 2205, the computer system may receive a procedure selection from the user. As previously discussed, such as selection may occur in a variety of manners. The user may, e.g., select the surgical dataset represented by one of panes 815, 820, 825, 830, 1030a, 1030b, 1030c, and 1030d, select a point on the scatter plot of the My Metrics window 1300 when in “visuals mode” as in FIG. 13, select a row in the table of the My Metrics window 1300 when in “table mode” as in FIG. 14, or select a point in the quick access metric map scatter plot of the Procedure View window 1700, as shown in FIG. 17.

In some embodiments, the path taken to the procedure window may affect the configuration of the various playback, task, and metrics panes. For example, at block 2210, the system may determine if a filter, such as a task filter, e.g., via filter drop-down pane 1105, filter drop-down pane 1205, task selection icon 1305b, etc. was active. Where no filter was selected, each of the playback, metric, and task panes may be set to the “default” configuration at block 2215a. For example, the playback pane may be set to the start of the video, the corresponding first task selected in each of the task panes and task rectangles, and the table of metrics displaying the left-most column (rather than focusing on any preselected metric) for the first task where no task selection was previously identified. In contrast, where a filter was selected, at block 2215b, the system may instead adjust one or more of the playback, task panes, rectangles, and metrics. For example, in addition to advancing playback to the selected task, where a specific metric was selected via drop-down 1305c before clicking upon a point in scatter plot 1340, the initial position of the table appearing in task-metrics region 1550 may be offset so as to present the column with that selected metric OPI value to the viewer. Thus, just as initially configuring the window for a task may improve the user's review, so may configuring the review for a specific metric facilitate comparison between surgeries. That is, a user interested in specific tasks or metrics may retain that focus even as they transition between different procedures. Thus, selection of a metric may result in pre-configuration of region 1550, but also the Y-axis of plot 1720.

Specifically, at block 2225, the computer system may determine the range for the metric map, e.g., the desired Y-axis of 1720. For example, where the user has selected neither a task nor a specific metric, then at block 2225, the metric map, such as scatter plot 1720, may be set to its “default” values, e.g., a scatter plot where the Y-axis is the total duration of the entire procedure (rather than the duration of a specific task). In contrast, where the user has filtered for only a task of interest, but has not specified a specific metric, then at block 2225 the Y-axis of the scatter plot may instead be the total duration of that task and include only points for procedures which include that task. Where the user has selected both a task and a metric, then at block 2225 the Y-axis of the scatter plot may be set to the values of that metric for that task and include only points for procedures which include that task. Finally, where a metric is selected, but not a task, then at block 2225 the Y-axis may be set to, e.g., the average values of that metric across all tasks containing that metric, the average value for that metric in the first task or for the currently depicted frame, etc. In this example, the points in the scatter plot may reflect only those procedures with a task associated with that metric. At block 2230, representations of related procedures may be presented on the page (e.g., points in scatter plot 1720, determination of metric values in row 1605 based on some or all of the related procedures, etc.). The related procedures may be the same as those in the metric map of the My Metrics page, the procedures presented in a previous iteration of the quick access metric map, those user procedures identified based upon previous filtering, etc.

In embodiments where the Procedure View window does not include an expert peer video playback (e.g., in window 1500), per the system configuration or user's choice, the system may transition from block 2235 to block 2255a (e.g., the user is transitioning to window 1500 after selecting pane 815, or a scatter plot point, without indicating any desire to view an expert video). In some embodiments, the presence of playback interface 1610 may be tied to the presence of the expert metrics row 1605, i.e., removal of interface 1610 likewise results in removal of row 1605, while introduction of the interface 1610 likewise causes row 1605 to be presented. Thus, such elements may be absent in the presentation at block 2255a. One will appreciate that in some embodiments the single surgery playback in window 1500 may be that of an expert surgery only, as when the user selects a recommended video panel in lieu of a user video. Similarly, where only a user or expert video applies, some embodiments may present window 1600 with only one of the two playbacks in operation.

Where the system instead determines that a peer contextual expert video is to be displayed at block 2235 (e.g., that is the system's default configuration, the user selected both a user and recommended video in an intermediate panel, etc.), the system may transition to block 2240. In some situations, the user may have explicitly identified, or the system may have already explicitly identified, a preferred expert video. For example, the user may have made a confirmation in an intermediate panel in region 1355, e.g., panel 2010. In these situations, an expert surgical procedure to be presented in the Procedure View window may be already known to the system, and so the system may transition from block 2240 to block 2245, using the identified procedure in the Procedure View window.

In contrast, where an expert playback is desired, but has not yet been explicitly identified, the system may then identify suitable procedures for playback, e.g., using the processes described herein with respect to the process 900, at blocks 2250a and 2250b. Accordingly, this recommendation may be determined based upon the procedure types, tasks, or metrics selected by the user or by those appearing in the selected procedure. For example, where the depicted procedure is a cholecystectomy, surgical datasets depicting expert performances of cholecystectomies may be included in the corpus. Process 900 may then operate upon this corpus. Similarly, where the user has filtered for a specific task or metric, then expert datasets with that task or metric may be included in the corpus. Having determined a corpus, and possibly applied process 900 thereto, the resulting elements may be ordered at block 2250b, e.g., in decreasing relevance.

In some embodiments, at block 2250c the most relevant dataset (e.g., the first in the ordering of block 2250b) may be the dataset whose video is presented in the peer video playback interface 1610, as, e.g., when the user specifies automatic expert playback. The system may then use the procedure dataset identified at block 2250c, along with the previously determined configuration items, in the presentation of the Procedure Window at block 2255b.

### Example OPI Table Listings

FIGS. 23-26 present example OPI metrics and example definitions, a description of each, and their relation to various skills and tasks as they may be used in the windows and interfaces discussed herein. As regards robotic arms, “SCE” refers to the “surgeon console”, “Cam” to the arm holding the camera, “D” the dominant arm of a robotic system, “N-D” to the non-dominant arm of the robotic system, and “Ret” refers to the retracting arm of the robot. As regards skills, “E” indicates “energy”, “S” refers to “suture”, “D” refers to “dissection”, “CU” refers to “camera use”, “AR” refers to “arm retraction”, “1-HD” refers to “1-hand dissection”, “2-HAR” refers to “2-hand arm retraction”. As regards tasks, “SL” indicates the “Suspensory Ligaments” task, “2-HS” indicates the “2-Hand Suture” task, “1-HS” indicates the “1-Hand Suture” task, “RS” refers to the “Running Suture” task, “UH” to the “Uterine Horn” task, and “RA/V” to the “Rectal Artery/Vein” task. Again, while skills are presented in these tables to facilitate understanding, one will appreciate that OPIs may be mapped to tasks directly without explicit consideration of any intervening skill.

### Computer System

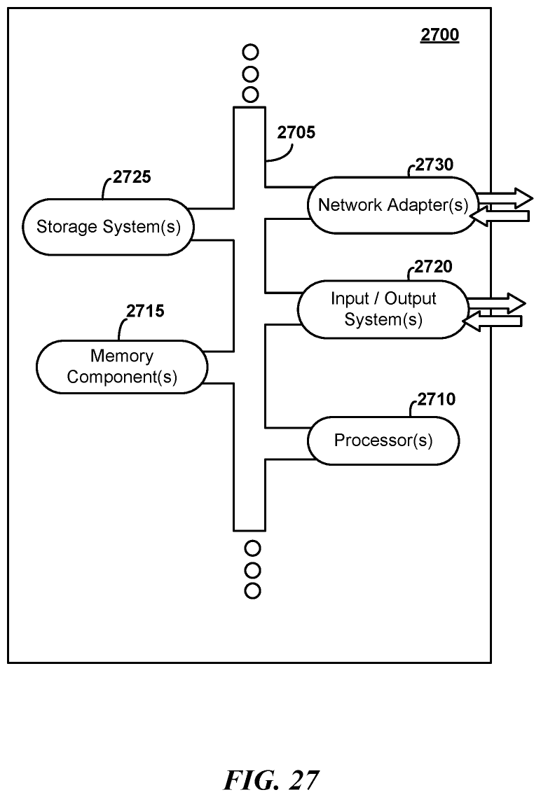

FIG. 27 is a block diagram of an example computer system as may be used in conjunction with some of the embodiments. The computing system 2700 may include an interconnect 2705, connecting several components, such as, e.g., one or more processors 2710, one or more memory components 2715, one or more input/output systems 2720, one or more storage systems 2725, one or more network adaptors 2730, etc. The interconnect 2705 may be, e.g., one or more bridges, traces, busses (e.g., an ISA, SCSI, PCI, I2C, Firewire bus, etc.), wires, adapters, or controllers.

The one or more processors 2710 may include, e.g., an Intel™ processor chip, a math coprocessor, a graphics processor, etc. The one or more memory components 2715 may include, e.g., a volatile memory (RAM, SRAM, DRAM, etc.), a non-volatile memory (EPROM, ROM, Flash memory, etc.), or similar devices. The one or more input/output devices 2720 may include, e.g., display devices, keyboards, pointing devices, touchscreen devices, etc. The one or more storage devices 2725 may include, e.g., cloud based storages, removable USB storage, disk drives, etc. In some systems memory components 2715 and storage devices 2725 may be the same components. Network adapters 2730 may include, e.g., wired network interfaces, wireless interfaces, Bluetooth™ adapters, line-of-sight interfaces, etc.

One will recognize that only some of the components, alternative components, or additional components than those depicted in FIG. 27 may be present in some embodiments. Similarly, the components may be combined or serve dual-purposes in some systems. The components may be implemented using special-purpose hardwired circuitry such as, for example, one or more ASICs, PLDs, FPGAs, etc. Thus, some embodiments may be implemented in, for example, programmable circuitry (e.g., one or more microprocessors) programmed with software and/or firmware, or entirely in special-purpose hardwired (non-programmable) circuitry, or in a combination of such forms.

In some embodiments, data structures and message structures may be stored or transmitted via a data transmission medium, e.g., a signal on a communications link, via the network adapters 2730. Transmission may occur across a variety of mediums, e.g., the Internet, a local area network, a wide area network, or a point-to-point dial-up connection, etc. Thus, “computer readable media” can include computer-readable storage media (e.g., “non-transitory” computer-readable media) and computer-readable transmission media.

The one or more memory components 2715 and one or more storage devices 2725 may be computer-readable storage media. In some embodiments, the one or more memory components 2715 or one or more storage devices 2725 may store instructions, which may perform or cause to be performed various of the operations discussed herein. In some embodiments, the instructions stored in memory 2715 can be implemented as software and/or firmware. These instructions may be used to perform operations on the one or more processors 2710 to carry out processes described herein. In some embodiments, such instructions may be provided to the one or more processors 2710 by downloading the instructions from another system, e.g., via network adapter 2730.

### Remarks

The drawings and description herein are illustrative. Consequently, neither the description nor the drawings should be construed so as to limit the disclosure. For example, titles or subtitles have been provided simply for the reader's convenience and to facilitate understanding. Thus, the titles or subtitles should not be construed so as to limit the scope of the disclosure, e.g., by grouping features which were presented in a particular order or together simply to facilitate understanding. Unless otherwise defined herein, all technical and scientific terms used herein have the same meaning as commonly understood by one of ordinary skill in the art to which this disclosure pertains. In the case of conflict, this document, including any definitions provided herein, will control. A recital of one or more synonyms herein does not exclude the use of other synonyms. The use of examples anywhere in this specification including examples of any term discussed herein is illustrative only, and is not intended to further limit the scope and meaning of the disclosure or of any exemplified term.

Similarly, despite the particular presentation in the figures herein, one skilled in the art will appreciate that actual data structures used to store information may differ from what is shown. For example, the data structures may be organized in a different manner, may contain more or less information than shown, may be compressed and/or encrypted, etc. The drawings and disclosure may omit common or well-known details in order to avoid confusion. Similarly, the figures may depict a particular series of operations to facilitate understanding, which are simply exemplary of a wider class of such collection of operations. Accordingly, one will readily recognize that additional, alternative, or fewer operations may often be used to achieve the same purpose or effect depicted in some of the flow diagrams. For example, data may be encrypted, though not presented as such in the figures, items may be considered in different looping patterns (“for” loop, “while” loop, etc.), or sorted in a different manner, to achieve the same or similar effect, etc.

Reference herein to “an embodiment” or “one embodiment” means that at least one embodiment of the disclosure includes a particular feature, structure, or characteristic described in connection with the embodiment. Thus, the phrase “in one embodiment” in various places herein is not necessarily referring to the same embodiment in each of those various places. Separate or alternative embodiments may not be mutually exclusive of other embodiments. One will recognize that various modifications may be made without deviating from the scope of the embodiments.

1-60. (canceled)

61. A computer-implemented method for presenting surgical performance data, the method comprising:
causing a first plurality of interface elements to be presented to a user in a first interface, each element of the first plurality of interface elements associated with at least one surgical dataset of a first plurality of surgical datasets;
receiving, from the user, a selection of an interface element from the first plurality of interface elements; and
in response, at least in part, to the user selection of the interface element from the first plurality of interface elements, causing a second interface to be presented to the user, the second interface comprising:
a playback region, the playback region depicting at least one video frame from the surgical dataset associated with the selected interface element; and
a second plurality of interface elements, each interface element of the second plurality of interface elements associated with at least one surgical dataset of a second plurality of surgical datasets.

62. The computer-implemented method of claim 61, wherein,
the first plurality of interface elements comprises a plurality of rows in a table of the first interface, and wherein,
the second plurality of interface elements comprises a plurality of scatter plot points in a scatter plot of the second interface.

63. The computer-implemented method of claim 62, wherein,
each row of the plurality of rows depicts metric values of at least two types of metrics for the surgical dataset associated with the row, wherein,
the scatter plot comprises:
a first axis indicating a range of metric values associated with a single type of metric of the at least two types of metrics; and
a second axis indicating acquisition times of each surgical dataset of the second plurality of surgical datasets, and wherein,
the method further comprises:
in response, at least in part, to a user selection of a scatter plot point, updating the playback region with data of the surgical dataset associated with the selected scatter plot point.

64. The computer-implemented method of claim 61, wherein,
both the first interface and the second interface present data associated with a same surgical task, and wherein,
depicting at least one video frame from the surgical dataset associated with the selected interface element comprises advancing to a video frame associated with a starting time of the same surgical task.

65. The computer-implemented method of claim 64, wherein,
the second interface depicts a plurality of metric values of a same type of metric, the metric values associated with each surgical dataset of the second plurality of surgical datasets, and wherein,
the second interface depicts a time of acquisition associated with each surgical dataset of the second plurality of surgical datasets.

66. The computer-implemented method of claim 65, wherein the method further comprises:
in response, at least in part, to a user selection of an interface element of the second plurality of interface elements, causing data of the surgical dataset associated with the selected interface element of the second plurality of interface elements to appear in the playback region; and
advancing the playback region to a video frame associated with a starting time of the same surgical task, and wherein,
the first plurality of surgical datasets and the second plurality of surgical datasets are the same plurality of surgical datasets.

67. The computer-implemented method of claim 66, wherein,
each metric value of the plurality of metric values of the same type of metric was derived, at least in part, from sensor data acquired from sensors present in a surgical theater.

68. A non-transitory computer-readable medium comprising instructions configured to cause a computer system to perform a method for presenting surgical performance data, the method comprising:
causing a first plurality of interface elements to be presented to a user in a first interface, each element of the first plurality of interface elements associated with at least one surgical dataset of a first plurality of surgical datasets;
receiving, from the user, a selection of an interface element from the first plurality of interface elements; and
in response, at least in part, to the user selection of the interface element from the first plurality of interface elements, causing a second interface to be presented to the user, the second interface comprising:
a playback region, the playback region depicting at least one video frame from the surgical dataset associated with the selected interface element; and
a second plurality of interface elements, each interface element of the second plurality of interface elements associated with at least one surgical dataset of a second plurality of surgical datasets.

69. The non-transitory computer-readable medium of claim 68, wherein,
the first plurality of interface elements comprises a plurality of rows in a table of the first interface, and wherein,
the second plurality of interface elements comprises a plurality of scatter plot points in a scatter plot of the second interface.

70. The non-transitory computer-readable medium of claim 69, wherein,
each row of the plurality of rows depicts metric values of at least two types of metrics for the surgical dataset associated with the row, wherein,
the scatter plot comprises:
a first axis indicating a range of metric values associated with a single type of metric of the at least two types of metrics; and
a second axis indicating acquisition times of each surgical dataset of the second plurality of surgical datasets, and wherein,
the method further comprises:
in response, at least in part, to a user selection of a scatter plot point, updating the playback region with data of the surgical dataset associated with the selected scatter plot point.

71. The non-transitory computer-readable medium of claim 68, wherein,
both the first interface and the second interface present data associated with a same surgical task, and wherein,
depicting at least one video frame from the surgical dataset associated with the selected interface element comprises advancing to a video frame associated with a starting time of the same surgical task.

72. The non-transitory computer-readable medium of claim 71, wherein,
the second interface depicts a plurality of metric values of a same type of metric, the metric values associated with each surgical dataset of the second plurality of surgical datasets, and wherein,
the second interface depicts a time of acquisition associated with each surgical dataset of the second plurality of surgical datasets.

73. The non-transitory computer-readable medium of claim 72, wherein the method further comprises:
in response, at least in part, to a user selection of an interface element of the second plurality of interface elements, causing data of the surgical dataset associated with the selected interface element of the second plurality of interface elements to appear in the playback region; and
advancing the playback region to a video frame associated with a starting time of the same surgical task, and wherein,
the first plurality of surgical datasets and the second plurality of surgical datasets are the same plurality of surgical datasets.

74. The non-transitory computer-readable medium of claim 73, wherein,
each metric value of the plurality of metric values of the same type of metric was derived, at least in part, from sensor data acquired from sensors present in a surgical theater.

75. A computer-system comprising:
at least one processor; and
at least one memory, the at least one memory comprising instructions configured to cause the computer system to perform a method for presenting surgical performance data, the method comprising:
causing a first plurality of interface elements to be presented to a user in a first interface, each element of the first plurality of interface elements associated with at least one surgical dataset of a first plurality of surgical datasets;
receiving, from the user, a selection of an interface element from the first plurality of interface elements; and
in response, at least in part, to the user selection of the interface element from the first plurality of interface elements, causing a second interface to be presented to the user, the second interface comprising:
a playback region, the playback region depicting at least one video frame from the surgical dataset associated with the selected interface element; and
a second plurality of interface elements, each interface element of the second plurality of interface elements associated with at least one surgical dataset of a second plurality of surgical datasets.

76. The computer system of claim 75, wherein,
the first plurality of interface elements comprises a plurality of rows in a table of the first interface, and wherein,
the second plurality of interface elements comprises a plurality of scatter plot points in a scatter plot of the second interface.

77. The computer system of claim 76, wherein,
each row of the plurality of rows depicts metric values of at least two types of metrics for the surgical dataset associated with the row, wherein,
the scatter plot comprises:
a first axis indicating a range of metric values associated with a single type of metric of the at least two types of metrics; and
a second axis indicating acquisition times of each surgical dataset of the second plurality of surgical datasets, and wherein,
the method further comprises:
in response, at least in part, to a user selection of a scatter plot point, updating the playback region with data of the surgical dataset associated with the selected scatter plot point.

78. The computer system of claim 75, wherein,
both the first interface and the second interface present data associated with a same surgical task, and wherein,
depicting at least one video frame from the surgical dataset associated with the selected interface element comprises advancing to a video frame associated with a starting time of the same surgical task.

79. The computer system of claim 78, wherein,
the second interface depicts a plurality of metric values of a same type of metric, the metric values associated with each surgical dataset of the second plurality of surgical datasets, and wherein,
the second interface depicts a time of acquisition associated with each surgical dataset of the second plurality of surgical datasets.

80. The computer system of claim 79, wherein the method further comprises:
in response, at least in part, to a user selection of an interface element of the second plurality of interface elements, causing data of the surgical dataset associated with the selected interface element of the second plurality of interface elements to appear in the playback region; and
advancing the playback region to a video frame associated with a starting time of the same surgical task, and wherein,
the first plurality of surgical datasets and the second plurality of surgical datasets are the same plurality of surgical datasets.
